(19) United States
(12) Patent Application Publication
(10) Pub. No.: US 2005/0043331 A1
(43) Pub. Date: Feb. 24, 2005

(54) Substituted 3-(2,5-disubstituted)pyridyl-4-aryl pyrroles for treating inflammatory diseases

(76) Inventors: James L. Bullington, Hamilton Square, NJ (US); Xiaodong Fan, Center Valley, PA (US); Paul F. Jackson, Whitehouse Station, NJ (US); Yue-Mei Zhang, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/670,031

(22) Filed: Sep. 24, 2003

Related U.S. Application Data
(60) Provisional application No. 60/414,436, filed on Sep. 27, 2002.

(51) Int. Cl. C07D 471/02; A61K 31/519; A61K 31/4745; A61K 31/4025

(52) U.S. Cl. 514/265.1; 514/300; 544/281; 546/113; 548/517; 514/422

(57) ABSTRACT

This invention provides novel substituted 3-(2,5-disubstituted)pyridyl-4-aryl pyrroles, and pharmaceutical compositions comprising same, useful for treating disorders ameliorated by reducing TNF-α production and/or p38 activity in appropriate cells. This invention also provides therapeutic and prophylactic methods using the instant pharmaceutical compositions.

### FIELD OF THE INVENTION

This invention relates to novel substituted 3-(2-substituted)pyridyl-4-aryl pyrroles and their therapeutic and prophylactic uses. Disorders treated and/or prevented using these compounds include inflammatory, emesis, anxiety, psychoses, anorexia, cognitive disorders, drug abuse and AIDS-related disorders.

### BACKGROUND OF THE INVENTION

TNF-α and p38-Related Disorders

Inflammatory cytokines such as TNF-α are produced via the activity of kinases. Such kinases include the Cytokine Suppressive Anti-inflammatory Drug-Binding Protein (CSBP)/p38 kinase, a Mitogen-Activated Protein (MAP) kinase family of serine-threonine protein kinases. Inflammatory cytokines play an important role in a number of inflammatory disorders (1), neurodegenerative disorders (10), and AIDS-related disorders (11-14). Although the precise mechanism of kinases such as p38 is unknown, p38 has been implicated in both the production of TNF-α and the signaling responses associated with the TNF-α receptor (6).

Arthritis is a prime example of an inflammatory disorder, and is thus the inflammatory disorder focused on most in this section. Arthritis affects millions of people and can strike at any joint in the human body. Its symptoms range from mild pain and inflammation in affected joints, to severe and debilitating pain and inflammation. Although the disorder is associated mainly with aging adults, it is not restricted to adults.

The most common rheumatoid arthritis therapy involves the use of nonsteroidal anti-inflammatory drugs (NSAID's) to alleviate symptoms. However, despite the widespread use of NSAID's, many individuals cannot tolerate the doses necessary to treat the disorder over a prolonged period of time. In addition, NSAID's merely treat the symptoms of disorder without affecting the underlying cause.

Other drugs such as methotrexate, gold salts, D-penicillamine and prednisone are often used when patients fail to respond to NSAID's. These drugs also have significant toxicities and their mechanism of action remains unknown. Monoclonal antibodies to TNF-α and receptor antagonists to interleukin 1β (IL-1β) have been shown to reduce symptoms of rheumatoid arthritis in small-scale human clinical trials (2).

In addition to protein-based therapies, there are small molecule agents that inhibit the production of these cytokines and have demonstrated activity in animal rheumatoid arthritis models (3). Of these small molecule agents, SB 203580 has proven effective in reducing the production of TNF-α and IL-1β in lipopolysaccharide (LPS)-stimulated human monocyte cell lines with IC₅₀ values of 50 to 100 nM (4).

In addition to in vitro testing results, SB 203580 has been shown to inhibit the production of inflammatory cytokines in rats and mice at IC₅₀ values of 15 to 25 mg/kg (5). SB 203580 reduces the production of inflammatory cytokines by inhibiting the activity of CSBP/p38 kinase at an IC₅₀ of 200 nM (6). Due to SB 203580's oral activity and potency in animal models, researchers have suggested that a compound with such an activity profile has potential as a viable treatment for rheumatoid arthritis (5).

Pyridylpyrroles and their analogs have also been prepared as cytokine inhibitors and glucagon antagonists (7), and specifically as inhibitors of IL-1β, TNF-α and other cytokines. Arylpyrroles (8) and triarylpyrroles (9) have also been prepared as cytokine inhibitors.

The role of CSBP/p38 has been implicated recently in various neurodegenerative and AIDS-related disorders. With regard to neurodegenerative disorders, p38 has been shown to have a role in determining whether a cell survives or undergoes neuronal programmed cell death or apoptosis (10, 11).

Also related to AIDS, the Kaposi's sarcoma-associated herpesvirus HHV8 has been shown to encode a G protein-coupled receptor that activates p38. It has been proposed that this activation contributes to tumorigenesis and angiogenesis leading to Kaposi's sarcoma (12).

Associated with AIDS is the rapid activation of p38 induced by infection of a CCR5⁺ human T cell line by SIV, suggesting that p38 may play a role in early viral infection (13). Additionally, p38 inhibitors have been shown to block HIV replication in vitro in a manner that may be TNF-α-independent (14).

Absence of Clinically Effective Agents

WO 00/33836 discloses 5-membered heterocycles stated to exhibit inhibitory activity against the selectins and are indicated in the treatment of human diseases involving selecting, some of which have the structure 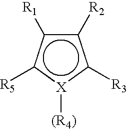
wherein substituents are as described in the reference.

WO 95/00501 discloses phenyl heterocycles as cyclooxygenase-2 inhibitors stated to have the structure 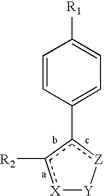
wherein substituents are as described in the reference.

WO 94/15932 discloses 3,4-diaryl thiophenes and analogs thereof stated to have use as anti-inflammatory agents, which have the structure 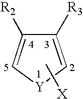
wherein substituents are as described in the reference.

WO 91/02730 discloses substituted five-membered heterocyclic rings stated to have the structure 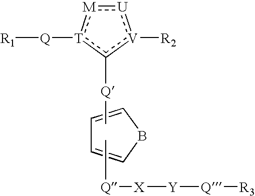
wherein substituents are as described in the reference.

In general, arthritis—particularly rheumatoid arthritis—and the host of other inflammatory and AIDS-related disorders all take a severe toll on those afflicted. There is a tremendous need for small molecule agents to treat these disorders. To date, however, no such agents have ever been identified and shown to be clinically effective in humans.

### SUMMARY OF THE INVENTION

This invention provides a compound having the structure 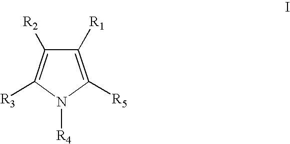
or a pharmaceutically acceptable salt thereof, wherein
R₁ and R₂ are independently selected from optionally substituted aryl and optionally substituted heteroaryl;
R₃ is selected from hydrogen, optionally substituted alkyl, —N═CR′″, —C(O)R′, —C(O)NR′R″, —NR′R″, optionally substituted aryl, optionally substituted heteroaryl, and optionally substituted heterocycle, wherein R′ and R″ are independently selected from hydrogen, optionally substituted alkyl, optionally substituted aryl, and optionally substituted heterocycle;
R₄ is selected from hydrogen, optionally substituted alkyl, optionally substituted aryl, optionally substituted heteroaryl, optionally substituted heterocycle, and —SiR′″R″″R′″″ wherein R′″, R″″, and R′″″ are each an independent straight chain or branched C₁₋₅alkyl, or R₃, R₄ and the —C—N— to which R₃ and R₄ are connected together form an optionally substituted 5- or 6-membered ring;
R₅ is selected from optionally substituted alkyl, —C(O)OR′, —C(O)NR′R″, C(O)NHNHC(O)R₆, —SO₂NR′R″, —C(O)R′, —NR′R″, nitrile, nitro, halo, and optionally substituted heterocycle, or R₄, R₅ and the —C—N— to which R₄ and R₅ are connected together form an optionally substituted 5- or 6-membered ring; and
R₆ is selected from H, alkyl, optionally substituted aryl;
with the provisos that
   (1) R₁ and R₂ are not both optionally substituted phenyl;
   (2) if either R₁ or R₂ is optionally substituted phenyl or 3-thienyl, and the other is unsubstituted 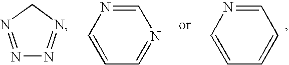
   then R₃ is not hydrogen, unsubstituted alkyl, —(CH₂)₃OH, —(CH₂)₃PH, —(CH₂)₃OMs, or —(CH₂)₂N(CH₂)₂O(CH₂)₂, and R₅ is not unsubstituted alkyl, —(CH₂)₃OH, —(CH₂)₃PH, —(CH₂)₃OMs, or —(CH₂)₂N(CH₂)₂O(CH₂)₂; and
   (3) R₄ does not form a fused ring with both R₃ and R₅.

This invention also provides a pharmaceutical composition comprising the instant compound and a pharmaceutically acceptable carrier.

This invention further provides a method of treating a subject having a disorder ameliorated by reducing TNF-α production and/or p38 activity in appropriate cells, which comprises administering to the subject a therapeutically effective dose of the instant pharmaceutical composition.

Finally, this invention provides a method of preventing an inflammatory response in a subject, comprising administering to the subject a prophylactically effective amount of the instant pharmaceutical composition either preceding or subsequent to an event anticipated to cause the inflammatory response in the subject.

### DETAILED DESCRIPTION OF THE INVENTION

This invention provides a compound having the structure 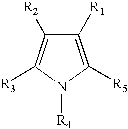
or a pharmaceutically acceptable salt thereof, wherein
R₁ and R₂ are independently selected from optionally substituted aryl and optionally substituted heteroaryl;
R₃ is selected from hydrogen, optionally substituted alkyl, —N═CR′″, —C(O)R′, —C(O)NR′R″, —NR′R″, optionally substituted aryl, optionally substituted heteroaryl, and optionally substituted heterocycle, wherein R′ and R″ are independently selected from hydrogen, optionally substituted alkyl, optionally substituted aryl, and optionally substituted heterocycle;
R₄ is selected from hydrogen, optionally substituted alkyl, optionally substituted aryl, optionally substituted heteroaryl, optionally substituted heterocycle, and —SiR′″R″″R′″″ wherein R′41 , R″″, and R′″″ are each an independent straight chain or branched C₁₋₅alkyl, or R₃, R₄ and the —C—N— to which R₃ and R₄ are connected together form an optionally substituted 5- or 6-membered ring;
R₅ is selected from optionally substituted alkyl, —C(O)OR′, —C(O)NR′R″, C(O)NHNHC(O)R₆, —SO₂NR′R″, —C(O)R′, —NR′R″, nitrile, nitro, halo, and optionally substituted heterocycle, or R₄, R₅ and the —C—N— to which R₄ and R₅ are connected together form an optionally substituted 5- or 6-membered ring; and
R₆ is selected from H, alkyl, optionally substituted aryl;
with the provisos that
   (1) R₁ and R₂ are not both optionally substituted phenyl;
   (2) if either R₁ or R₂ is optionally substituted phenyl or 3-thienyl, and the other is unsubstituted 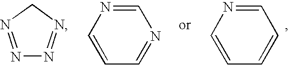
   then R₃ is not hydrogen, unsubstituted alkyl, —(CH₂)₃OH, —(CH₂)₃PH, —(CH₂)₃OMs, or —(CH₂)₂N(CH₂)₂O(CH₂)₂, and R₅ is not unsubstituted alkyl, —(CH₂)₃OH, —(CH₂)₃PH, —(CH₂)₃OMs, or —(CH₂)₂N(CH₂)₂O(CH₂)₂; and
   (3) R₄ doesn't form a fused ring with both R₃ and R₅.

Preferably, R₁ is substituted with one or more groups selected from hydrogen, amino, alkyl substituted amino, aryl substituted amino, hydroxy, methoxy, phenyl ether, S-alkyl, halogen, trifluoromethyl, and nitro.

Preferably, R₂ is substituted with one or more groups selected from hydrogen, amino, alkyl substituted amino, aryl substituted amino, hydroxy, methoxy, phenyl ether, S-alkyl, halogen, trifluoromethyl, and nitro. More preferably, R₂ is heteroaryl having 1-3 N.

Preferably, R₃ is selected from hydrogen, alkyl, aryl, heteroaryl, heterocycle, and —NR′R″, wherein R′ and R″ are independently selected from hydrogen, alkyl, aryl, and heterocycle.

Preferably, R₄ is hydrogen or alkyl. More preferably, R₄ is hydrogen or methyl.

Preferably, R₅ is selected from alkyl, —C(O)OR′, —C(O)NR′R″, nitrile, and heterocycle. In particular, the preferred alkyl is selected from —(CH₂)ₙOR′, —(CH₂)ₙNR′R′″, —(CH₂)ₙCOOR′, and —(CH₂)ₙCONR′R″; the preferred NR′ R″ group is —NHCOR′; the preferred heterocycles are ester isosteres (e.g. oxadiazole and the like, such as derivatives of 1,2,4-triazole, 1,2,4-triazol-3-ol, isoxazol-3-ol, imidazolidine-2,4-dione, 4H-[1,2,4]oxadiazol-5-one, 4H-[1,2,4]thiadiazol-5-one, 4H-[1,2,4]oxadiazole-5-thione, oxazole, [1,3,4]oxadiazole).

In a preferred embodiment, the compound is selected from the group of compounds shown in Table 1.

| | | | P38 | Mouse | Mouse |
|---|---|---|---|---|---|
| | PBMC cell | P38 cell | enzyme | 10 mg/kg % | 10 mg/kg % |
| Compound No. | IC_{50 nM} | 1 μM LJ | IC₅₀ nM | inhib 0.5 h | inhib 2 h |
| 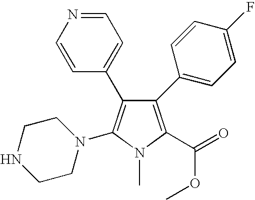 | 33 | | 10000 | 73 | |
| (Cpd 8) | | | | | |
| 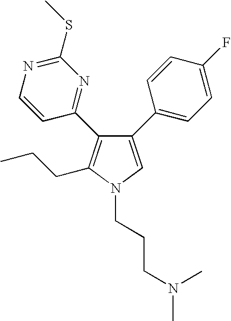 | 148 | | | (−37) | |
| (Cpd 48) | | | | | |
| 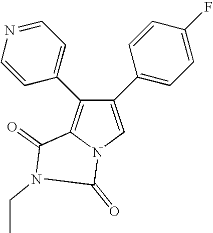 | 401 | | | 72 | |
| (Cpd 31) | | | | | |
| 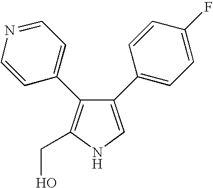 | 101 | | | (−72) | |
| (Cpd 26) | | | | | |
| 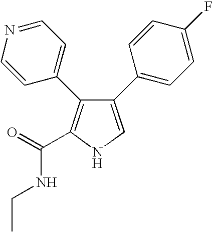 | 209 | | | (−24) | |
| (Cpd 32) | | | | | |
| 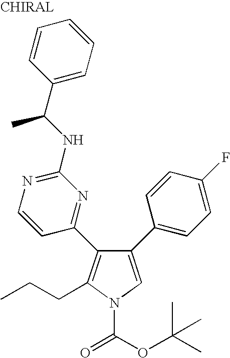 | | | | −79 | |
| (Cpd 49) | | | | | |
| 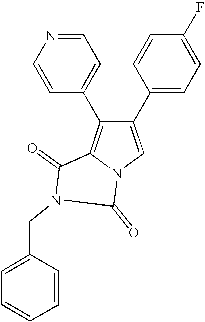 | 26 | | >10000 | 51 | |
| (Cpd 50) | | | | | |
| 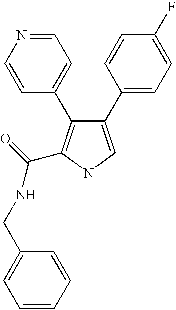 | 14 | | >10000 | (−60) | |
| (Cpd 51) | | | | | |
| 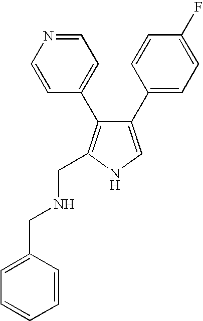 | | | | | |
| (Cpd 52) | | | | | |
| 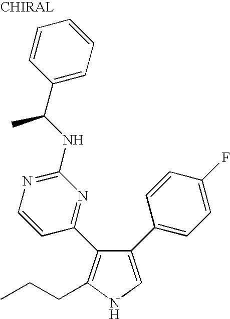 | 10 | | 1436 | −19 | |
| (Cpd 53) | | | | | |
| 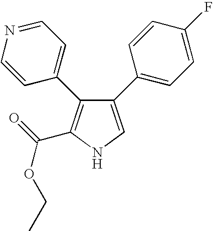 | 145 | | “>10,000” | | |
| (Cpd 54) | | | | | |
| 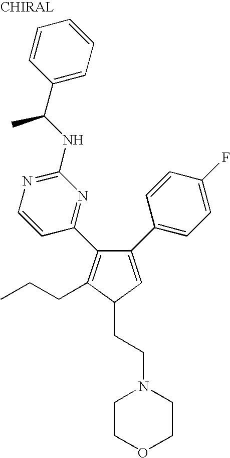 | 4 | | 10,000 | (−35) | |
| (Cpd 55) | | | | | |
| 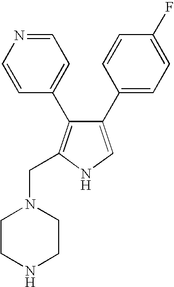 | 63 | | 856 | (−41) | |
| (Cpd 56) | | | | | |
| 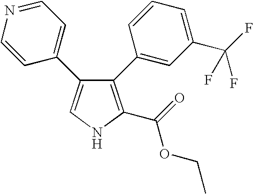 | 96 | | | −8 | |
| (Cpd 57) | | | | | |
| 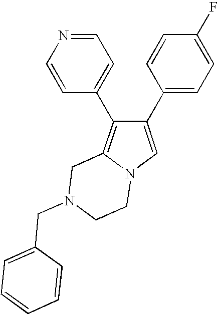 | 11 | | 1662 | −6 | |
| (Cpd 29) | | | | | |
| 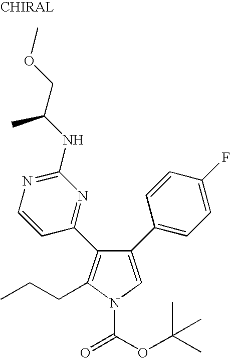 | 50 | | 1347 | | |
| (Cpdd 58) | | | | | |
| 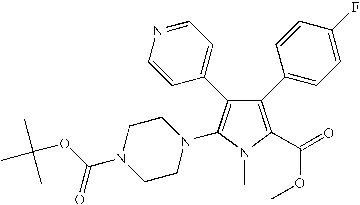 | 4259 | 28 | | | |
| (Cpd 8a) | | | | | |
| 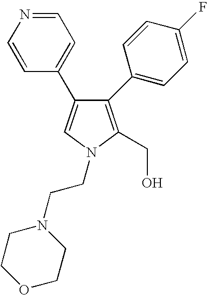 | 277 | 4 | 16 | −50 | |
| (Cpd 59) | | | | | |
| 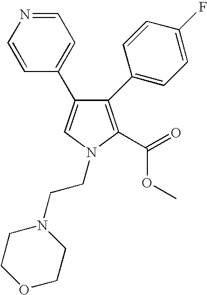 | 10000 | 1 | | | |
| (Cpd 60) | | | | | |
| 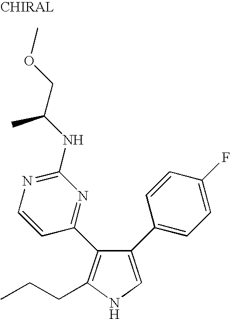 | 86 | 91 | 5 | (−35) | |
| (Cpd 61) | | | | | |
| 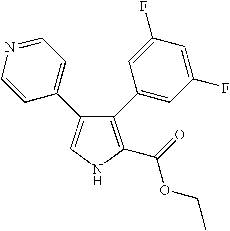 | 114 | 0 | 3474 | (−14) | |
| (Cpd 62) | | | | | |
| 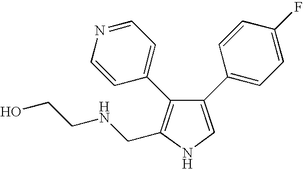 | 148 | 8 | >2000 | | |
| (Cpd 63) | | | | | |
| 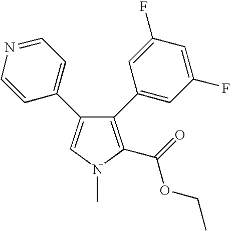 | 33 | 7 | 8687 | −31 | |
| (Cpd 64) | | | | | |
| 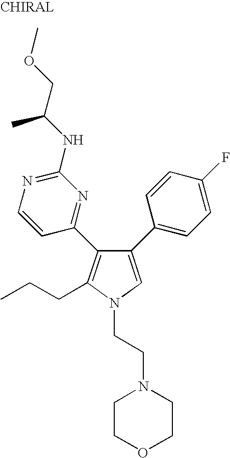 | 1 | 102 | 21 | −1 | |
| (Cpd 65) | | | | | |
| 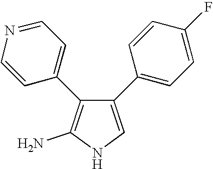 | 905 | 6 | 905 | | |
| (Cpd 66) | | | | | |
| 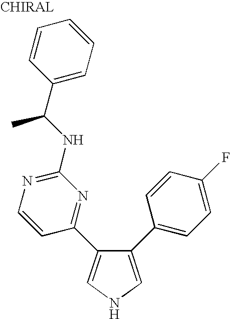 | 3 | 90 | 55 | | |
| (Cpd 67) | | | | | |
| 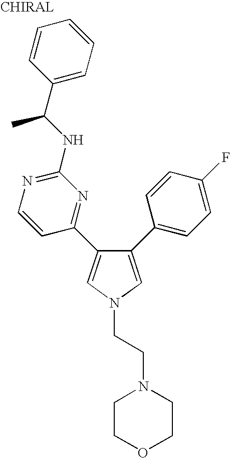 | 4 | 83 | 7 | −21 | |
| (Cpd 68) | | | | | |
| 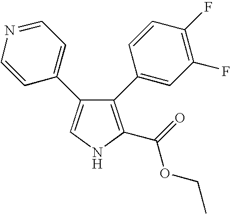 | 560 | 17 | >10000 | −32 | |
| (Cpd 69) | | | | | |
| 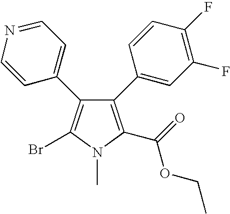 | 2721 | 12 | >10000 | −36 | |
| (Cpd 70) | | | | | |
| 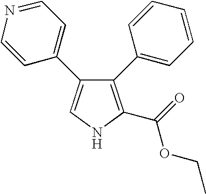 | 149 | 9 | 572 | 58 | |
| (Cpd 71) | | | | | |
| 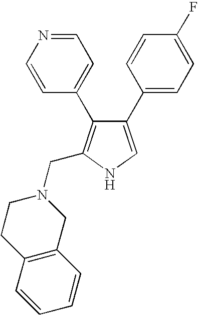 | 40 | 32 | 2000 | 63 | |
| (Cpd 72) | | | | | |
| 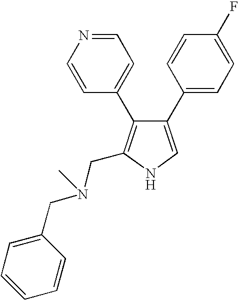 | 91 | 78 | | 37 | |
| (Cpd 73) | | | | | |
| 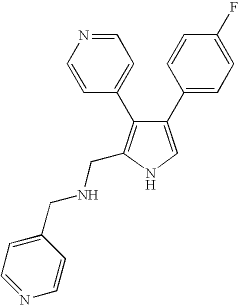 | 74 | 25 | 400 | 47 | |
| (Cpd 74) | | | | | |
| 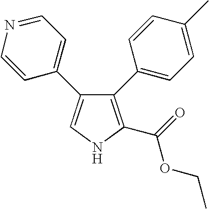 | 136 | 42 | | −218 | |
| (Cpd 75) | | | | | |
| 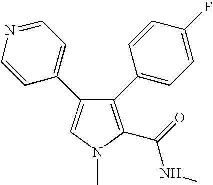 | 1478 | 18 | | 67 | 78 |
| (Cpd 76) | | | | | |
| 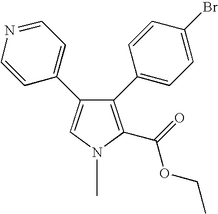 | 211 | 62 | | | |
| (Cpd 77) | | | | | |
| 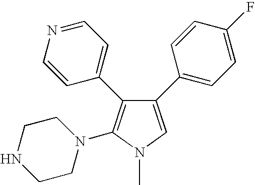 | 15 | 61 | 2024 | 75 | |
| (Cpd 78) | | | | | |
| 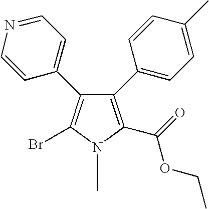 | 736 | 9 | | (−76) | |
| (Cpd 79) | | | | | |
| 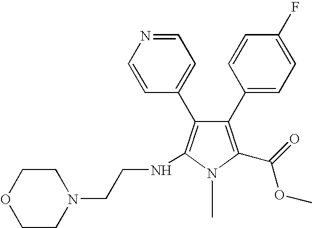 | 50 | 2 | >10,000 | 44 | |
| (Cpd 5) | | | | | |
| 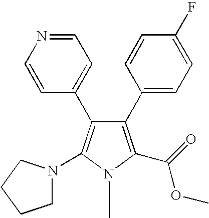 | 17 | 32 | 10,000 | −38 | |
| (Cpd 80) | | | | | |
| 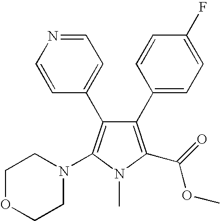 | 47 | 12 | 10,000 | −41 | |
| (Cpd 81) | | | | | |
| 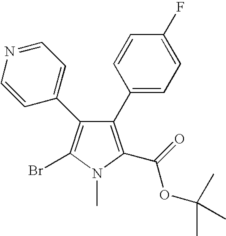 | 1890 | 8 | | −7 | |
| (Cpd 82) | | | | | |
| 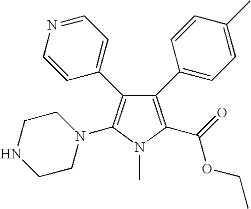 | 60 | 53 | 3939 | 79 | |
| (Cpd 83) | | | | | |
| 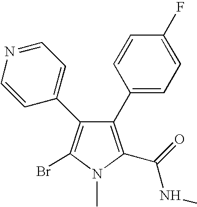 | 543 | 11 | | 53 | |
| (Cpd 84) | | | | | |
| 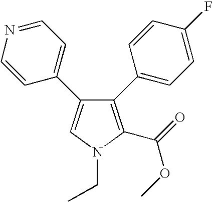 | 108 | 4 | | 53 | |
| (Cpd 85) | | | | | |
| 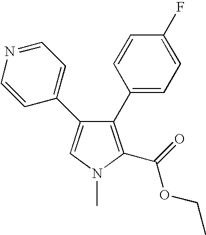 | 125 | −2 | | | |
| (Cpd 86) | | | | | |
| 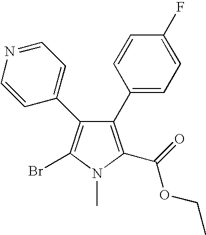 | 86 | 4 | | | |
| (Cpd 87) | | | | | |
| 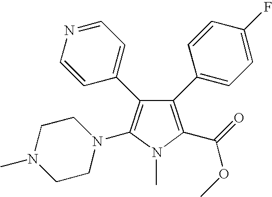 | 95 | 1 | 2000 | 89 | |
| (Cpd 10) | | | | | |
| 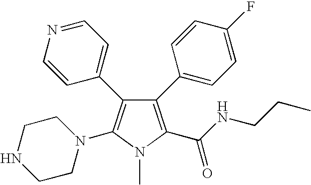 | 10000 | 7 | 10,000 | | |
| (Cpd 88) | | | | | |
| 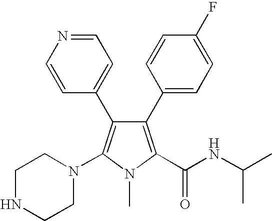 | | 22 | | | |
| (Cpd 89) | | | | | |
| 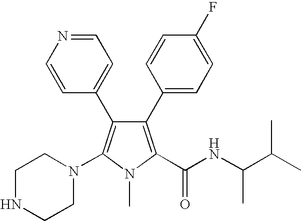 | | 15 | | | |
| (Cpd 90) | | | | | |
| 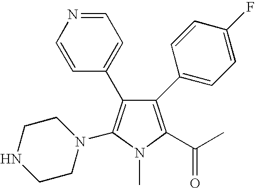 | 40 | 29 | 10,000 | 39 | |
| (Cpd 91) | | | | | |
| 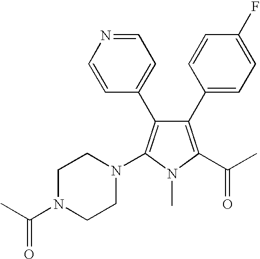 | 43 | 6 | 2000 | 61 | 28 |
| (Cpd 92) | | | | | |
| 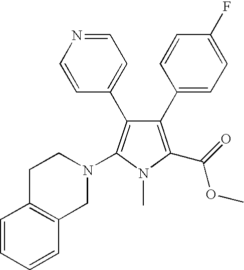 | 66 | 20 | >10,000 | 32 | |
| (Cpd 93) | | | | | |
| 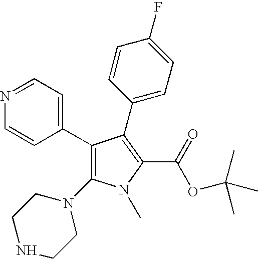 | 420 | 10 | | 25 | |
| (Cpd 94) | | | | | |
| 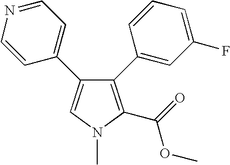 | 64 | 18 | | 52 | 47 |
| (Cpd 3) | | | | | |
| 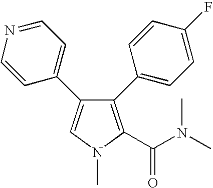 | 1383 | 21 | | 33 | |
| (Cpd 95) | | | | | |
| 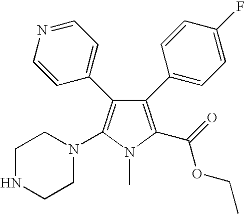 | 16 | 22 | >10,000 | 79 | |
| (Cpd 96) | | | | | |
| 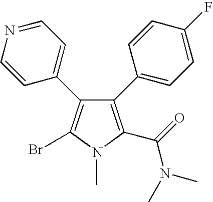 | 301 | 1 | | −41 | |
| (Cpd 97) | | | | | |
| 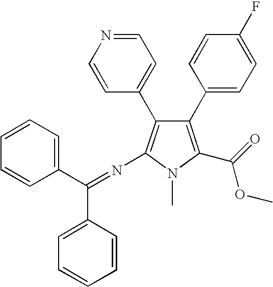 | 52 | −2 | | −49 | |
| (Cpd 98) | | | | | |
| 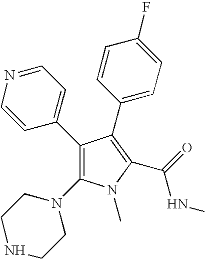 | 106 | 7 | >10,000 | (−7) | |
| (Cpd 99) | | | | | |
| 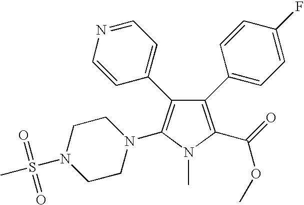 | 5861 | | | −29 | |
| (Cpd 12) | | | | | |
| 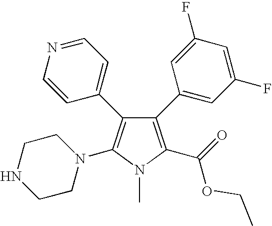 | 93 | 12 | >10,000 | 75 | |
| (Cpd 100) | | | | | |
| 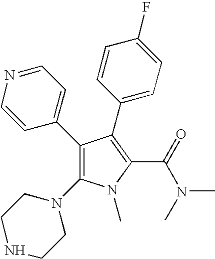 | 120 | 13 | >10,000 | | |
| (Cpd 101) | | | | | |
| 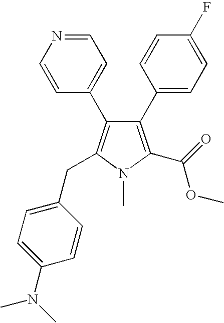 | 118 | 12 | >10,000 | 15 | |
| (Cpd 102) | | | | | |
| 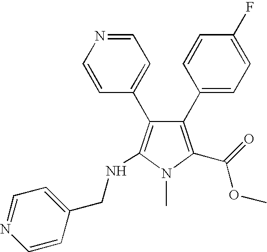 | 145 | 20 | >10,000 | −22 | |
| (Cpd 103) | | | | | |
| 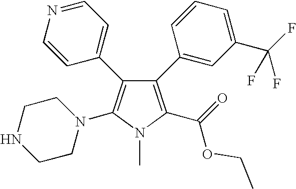 | 21 | 11 | >10,000 | 83 | |
| (Cpd 104) | | | | | |
| 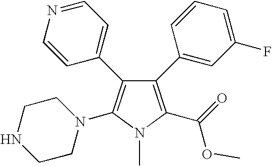 | 69 | 31 | >10,000 | 49 | |
| (Cpd 105) | | | | | |
| 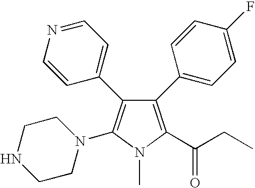 | 67 | 21 | >10,000 | 32 | |
| (Cpd 106) | | | | | |
| 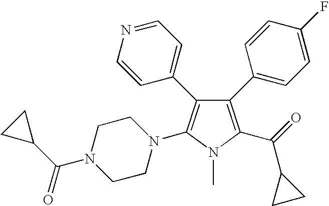 | 36 | 11 | 10,000 | | |
| (Cpd 107) | | | | | |
| 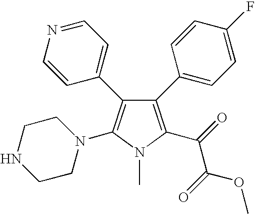 | 28 | 15 | 10,000 | 52 | |
| (Cpd 108) | | | | | |
| 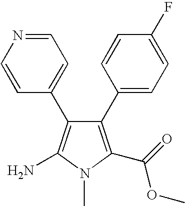 | 51 | 12 | 10,000 | 53.9 | |
| (Cpd 9) | | | | | |
| 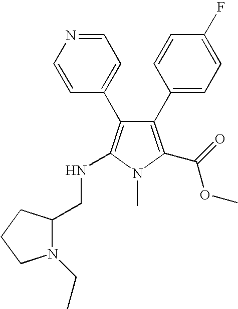 | 7752 | | 6178 | | |
| (Cpd 109) | | | | | |
| 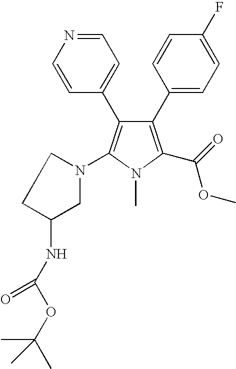 | 1660 | | | | |
| (Cpd 110) | | | | | |
| 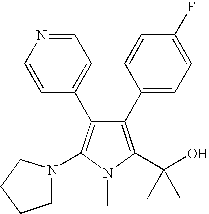 | 192 | | 2000 | 37.7 | |
| (Cpd 111) | | | | | |
| 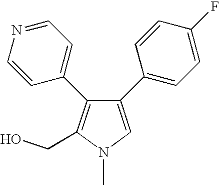 | 169 | | | 65 | |
| (Cpd 112) | | | | | |
| 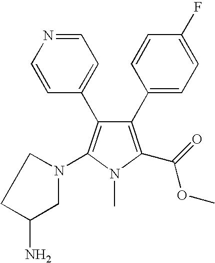 | 237 | | | 25 | |
| (Cpd 113) | | | | | |
| 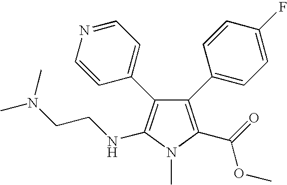 | 98 | | | (−43) | |
| (Cpd 114) | | | | | |
| 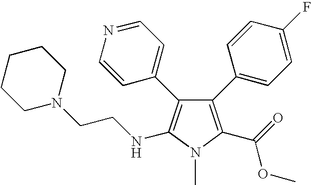 | 92 | | | (−36) | |
| (Cpd 115) | | | | | |
| 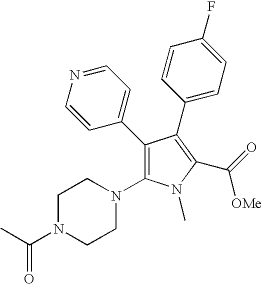 | 1531 | | | 57 | |
| (Cpd 116) | | | | | |
| 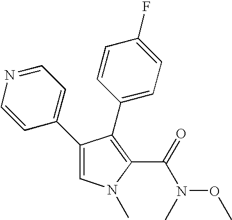 | 5838 | | | | |
| (Cpd 37) | | | | | |
| 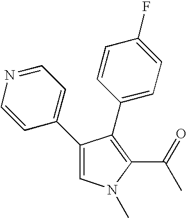 | 597 | | | | |
| (Cpd 39) | | | | | |
| 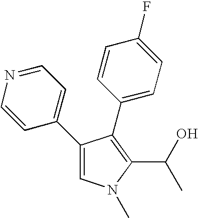 | 1452 | | | | |
| (Cpd 40) | | | | | |
| 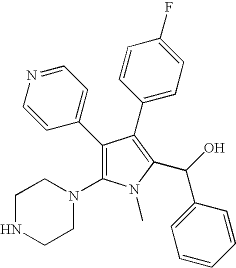 | 660 | | | | |
| (Cpd 117) | | | | | |
| 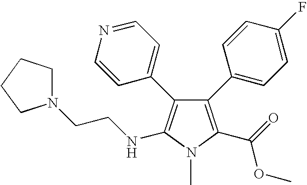 | 1393 | | | | |
| (Cpd 118) | | | | | |
| 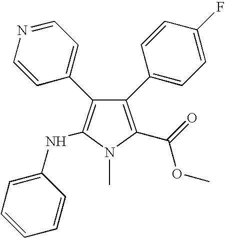 | 57 | | | −10.5 | |
| (Cpd 119) | | | | | |
| 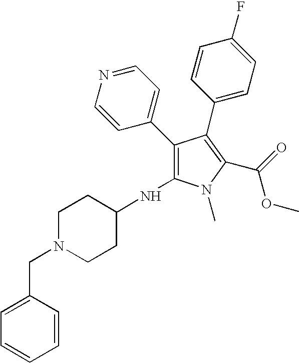 | 1307 | | | | |
| (Cpd 120) | | | | | |
| 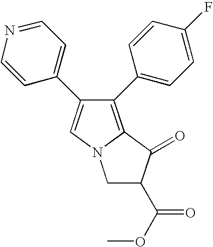 | 29 | | | 44.7 | |
| (Cpd 21) | | | | | |
| 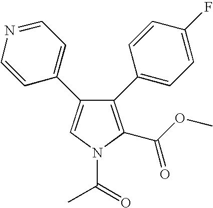 | 194 | | | −32.1 | |
| (Cpd 121) | | | | | |
| 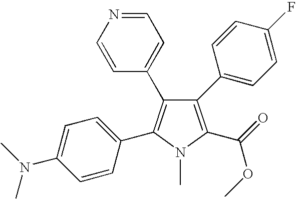 | 120 | | | −22 | |
| (Cpd 122) | | | | | |
| 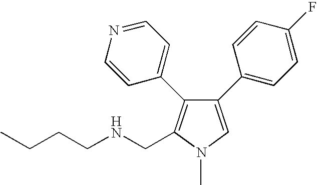 | 1085 | | | | |
| (Cpd 123) | | | | | |
| 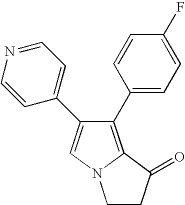 | 138 | | | 63 | −3 |
| (Cpd 22) | | | | | |
| 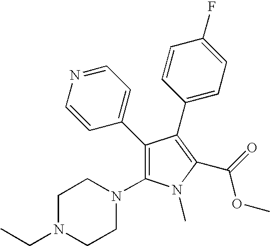 | 135 | | | 67.8 | |
| (Cpd 124) | | | | | |
| 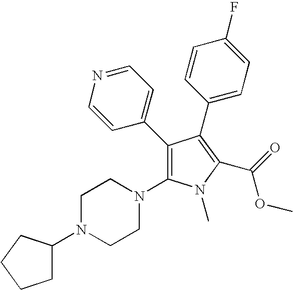 | 139 | | | 48.6 | |
| (Cpd 125) | | | | | |
| 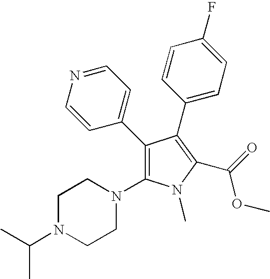 | 1981 | | | | |
| (Cpd 126) | | | | | |
| 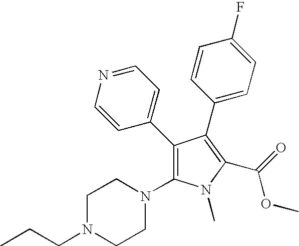 | 901 | | | | |
| (Cpd 127) | | | | | |
| 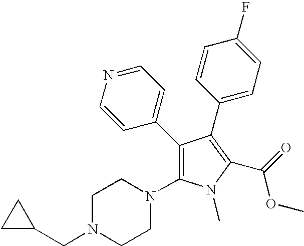 | 269 | | | 66.3 | |
| (Cpd 128) | | | | | |
| 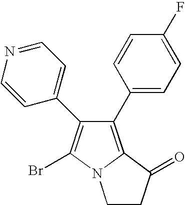 | 819 | | | 31 | |
| (Cpd 129) | | | | | |
| 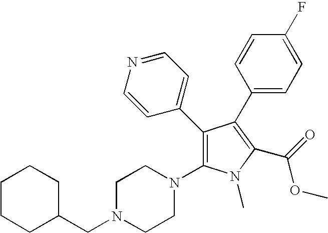 | 573 | | | | |
| (Cpd 130) | | | | | |
| 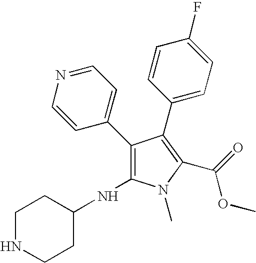 | 315 | | | 7 | |
| (Cpd 131) | | | | | |
| 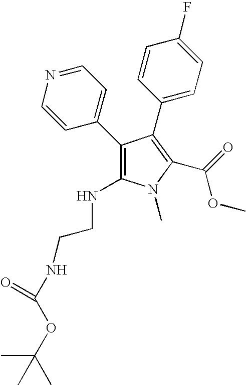 | 1635 | | | | |
| (Cpd 132) | | | | | |
| 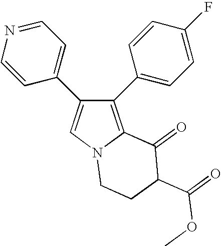 | 326 | | | −9 | |
| (Cpd 133) | | | | | |
| 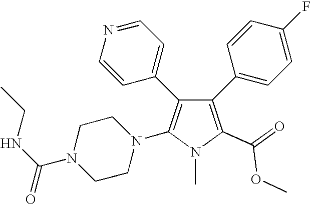 | 2922 | | | −38 | |
| (Cpd 13) | | | | | |
| 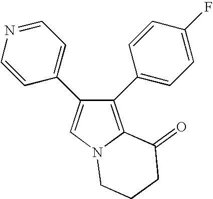 | 150 | | | 61 | 31 |
| (Cpd 134) | | | | | |
| 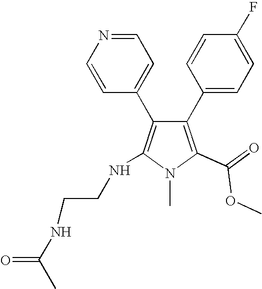 | 1689 | | | | |
| (Cpd 135) | | | | | |
| 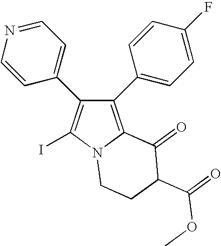 | | | | 15 | |
| (Cpd 136) | | | | | |
| 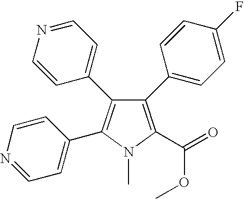 | | | | 51 | |
| (Cpd 137) | | | | | |
| 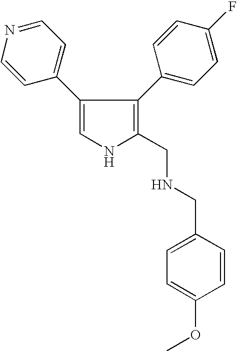 | 190 | | | −30 | |
| (Cpd 138) | | | | | |
| 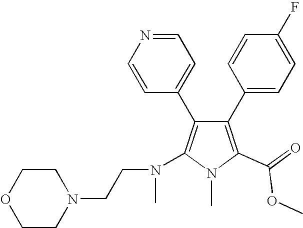 | | | | 66 | |
| (Cpd 11) | | | | | |
| 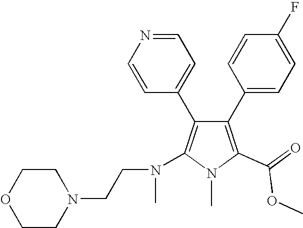 | 38 | | >10000 | 78 | |
| (Cpd 11) | | | | | |
| 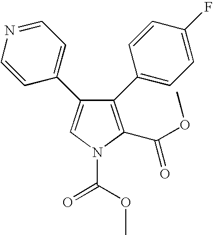 | 23 | | | −22 | |
| (Cpd 139) | | | | | |
| 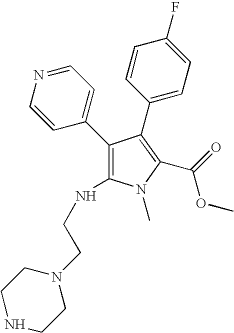 | | | | 77 | |
| (Cpd 140) | | | | | |
| 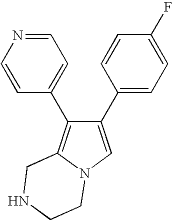 | 208 | | | 71 | |
| (Cpd 30) | | | | | |
| 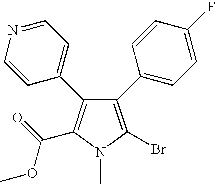 | 180 | | | −6 | |
| (Cpd 43) | | | | | |
| 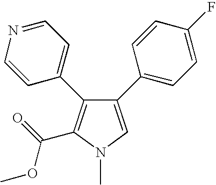 | 55 | | | −56 | |
| (Cpd 141) | | | | | |
| 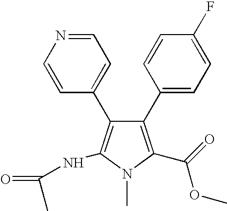 | 1493 | | | | |
| (Cpd 142) | | | | | |
| 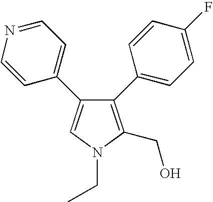 | 70 | | | 48 | |
| (Cpd 143) | | | | | |
| 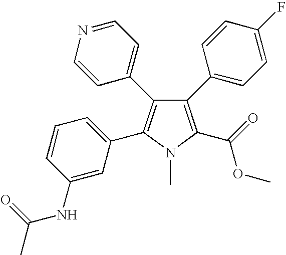 | 2 | | | 59 | |
| (Cpd 144) | | | | | |
| 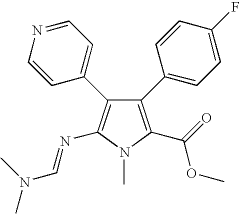 | 5 | | | 6 | |
| (Cpd 15) | | | | | |
| 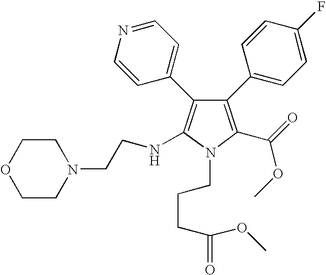 | 29 | | | | |
| (Cpd 23a) | | | | | |
| 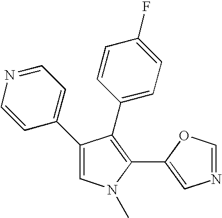 | 59 | | | 53 | |
| (Cpd 41) | | | | | |
| 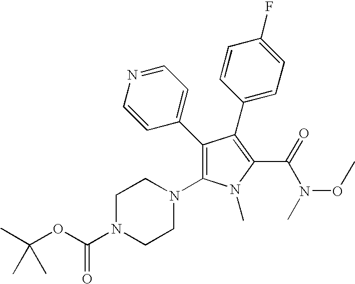 | 72 | | | 45 | |
| (Cpd 145) | | | | | |
| 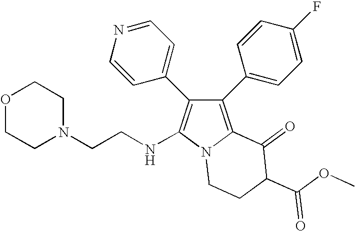 | 77 | | | 71 | |
| (Cpd 23) | | | | | |
| 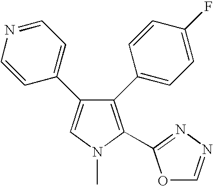 | 18 | | | 60 | −1 |
| (Cpd 35) | | | | | |
| 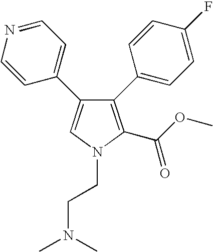 | | | | −32 | |
| (Cpd 146) | | | | | |
| 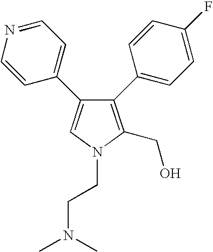 | 852 | | | 20 | |
| (Cpd 147) | | | | | |
| 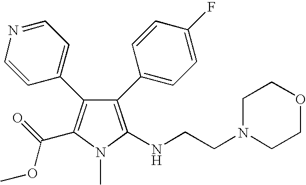 | 10000 | | | 91 | |
| (Cpd 148) | | | | | |
| 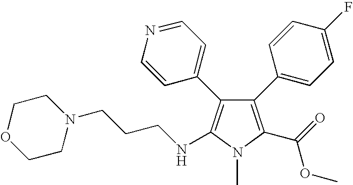 | | | | 64 | |
| (Cpd 149) | | | | | |
| 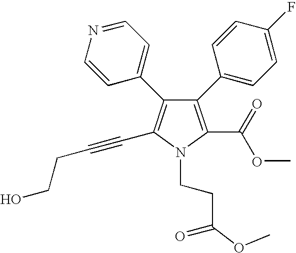 | 5284 | | | | |
| (Cpd 150) | | | | | |
| 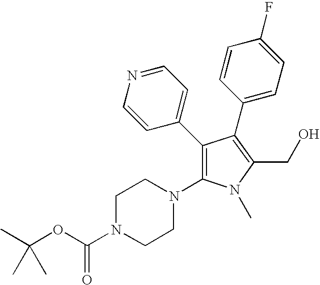 | | | | 83 | |
| (Cpd 151) | | | | | |
| 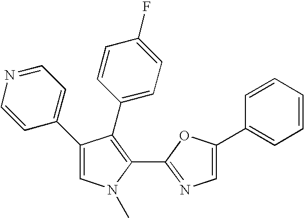 | 1030 | | | 81 | 35 |
| (Cpd 36) | | | | | |
| 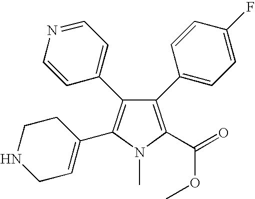 | 72 | | | 96 | |
| (Cpd 7) | | | | | |
| 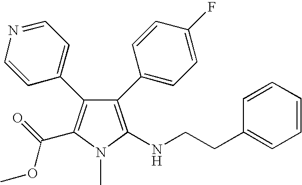 | 1022 | | | 58 | |
| (Cpd 152) | | | | | |
| 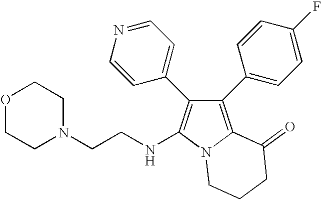 | 2559 | | | 51 | |
| (Cpd 24) | | | | | |
| 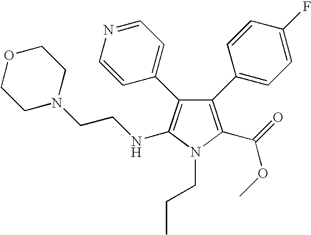 | 589 | | | | |
| (Cpd 153) | | | | | |
| 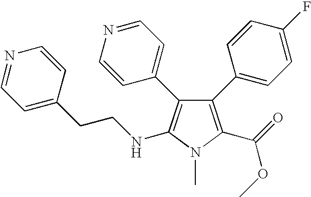 | 145 | | | | |
| (Cpd 154) | | | | | |
| 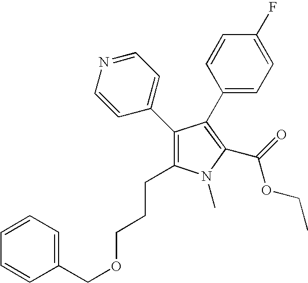 | 248 | | | 51 | |
| (Cpd 155) | | | | | |
| 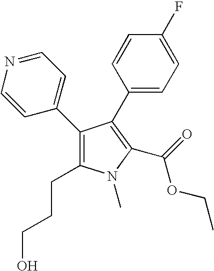 | 186 | | | | |
| (Cpd 17) | | | | | |
| 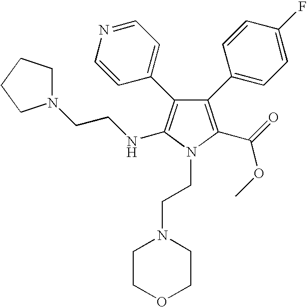 | 969 | | | 23 | |
| (Cpd 156) | | | | | |
| 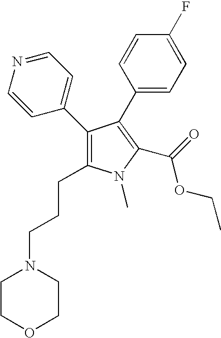 | 756 | | | 34 | |
| (Cpd 19) | | | | | |
| 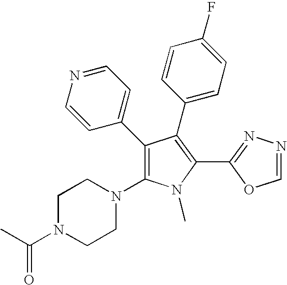 | | | | 19 | |
| (Cpd 157) | | | | | |
| 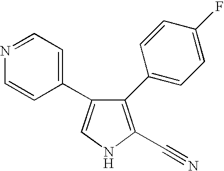 | 47 | | | | |
| (Cpd 158) | | | | | |
| 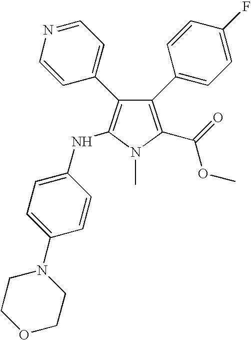 | 1556 | | | 29 | |
| (Cpd 159) | | | | | |
| 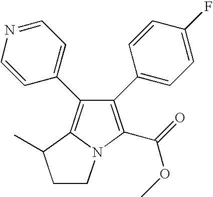 | 208 | | | 67 | 52 |
| (Cpd 47) | | | | | |
| 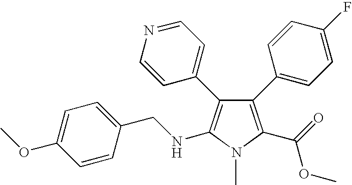 | 42 | | | 79 | −46 |
| (Cpd 160) | | | | | |
| 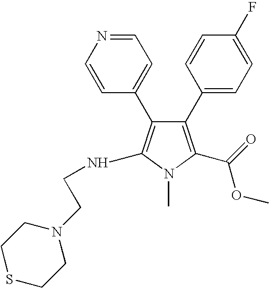 | 69 | | | | |
| (Cpd 161) | | | | | |
| 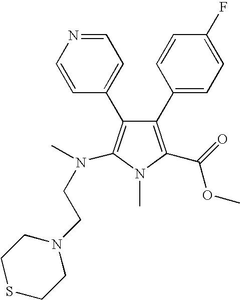 | 183 | | | | |
| (Cpd 162) | | | | | |
| 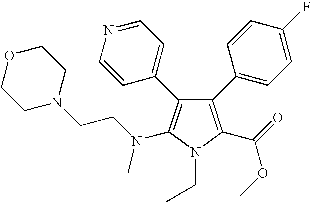 | 105 | | | 66 | 13 |
| (Cpd 163) | | | | | |
| 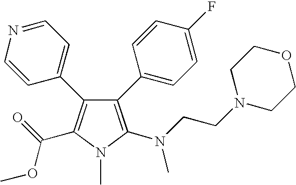 | 3514 | | | | |
| (Cpd 164) | | | | | |
| 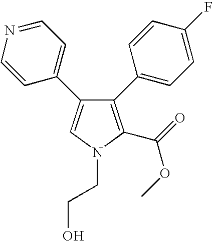 | 692 | | | | |
| (Cpd 165) | | | | | |
| 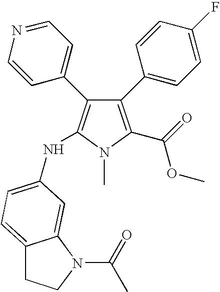 | 650 | | | | |
| (Cpd 166) | | | | | |
| 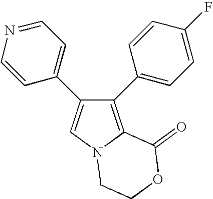 | 160 | | | | |
| (Cpd 46) | | | | | |
| 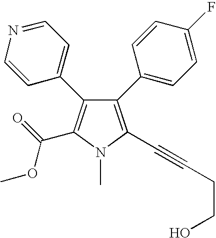 | 894 | | | 30 | |
| (Cpd 167) | | | | | |
| 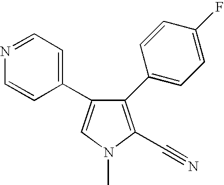 | 171 | | | 67 | −19 |
| (Cpd 168) | | | | | |
| 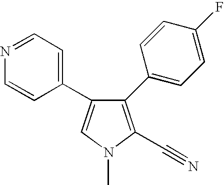 | | | | | 49 |
| (Cpd 169) | | | | | |
| 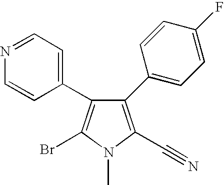 | 625 | | | 22 | |
| (Cpd 170) | | | | | |
| 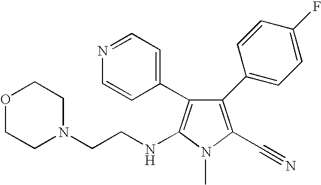 | 351 | | | | |
| (Cpd 171) | | | | | |
| 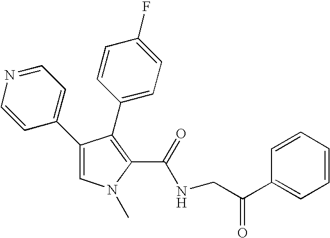 | 157 | | | 81 | 37 |
| (Cpd 34) | | | | | |
| 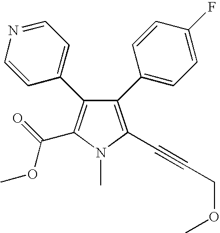 | 443 | | | −5 | |
| (Cpd 172) | | | | | |
| 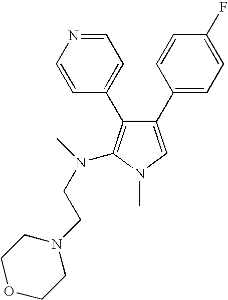 | 117 | | | 65 | −17 |
| (Cpd 173) | | | | | |
| 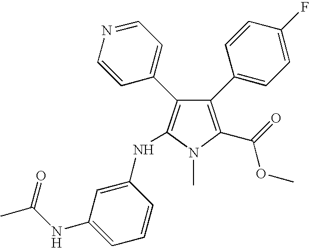 | 21 | | | | |
| (Cpd 174) | | | | | |
| 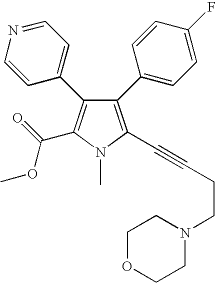 | 32 | | | | −4 |
| (Cpd 175) | | | | | |
| 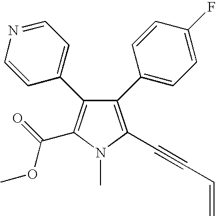 | 34 | | | | −20 |
| (Cpd 176) | | | | | |
| 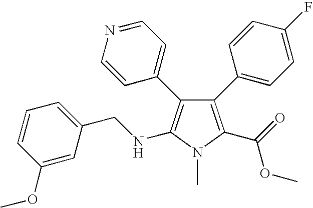 | 407 | | | | 63 |
| (Cpd 177) | | | | | |
| 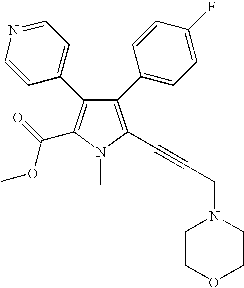 | 788 | | | | 47 |
| (Cpd 44) | | | | | |
| 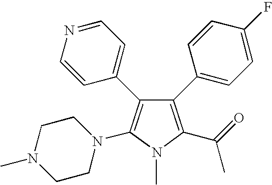 | 370 | | | | 40 |
| (Cpd 178) | | | | | |
| 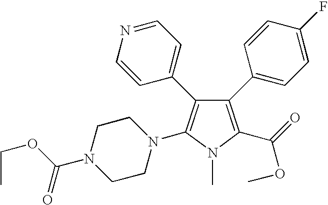 | 30 | | | | 8 |
| (Cpd 179) | | | | | |
| 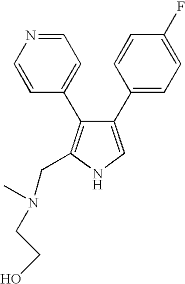 | 3 | | | | −8 |
| (Cpd 180) | | | | | |
| 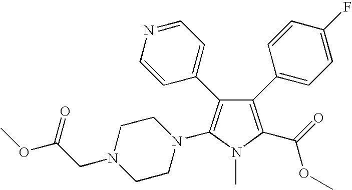 | 5 | | | | 26 |
| (Cpd 181) | | | | | |
| 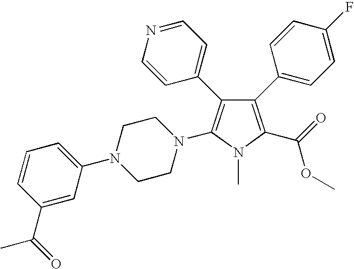 | 8 | | | | −21 |
| (Cpd 15) | | | | | |
| 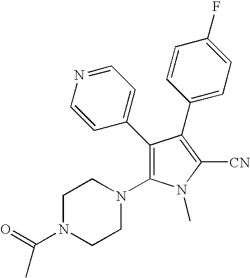 | 154 | | 2000 | | 81 |
| (Cpd 182) | | | | | |
| 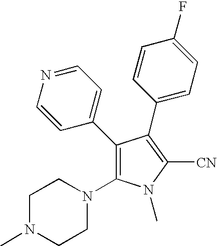 | 80 | | 2000 | | 47 |
| (Cpd 183) | | | | | |
| 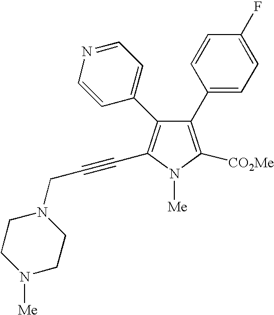 | 7.7 | | | | 57 |
| (Cpd 184) | | | | | |
| 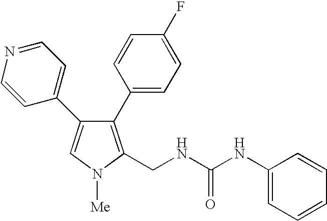 | 27 | | | | 82 |
| (Cpd 185) | | | | | |
| 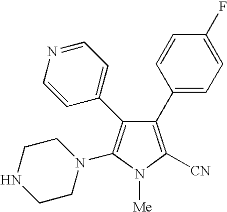 | 18 | | | | 67 |
| (Cpd 186) | | | | | |

The instant compounds can be isolated and used as free bases. They can also be isolated and used as pharmaceutically acceptable salts. Examples of such salts include hydrobromic, hydroiodic, hydrochloric, perchloric, sulfuric, maleic, fumaric, malic, tartaric, citric, benzoic, mandelic, methanesulfonic, hydroethanesulfonic, benzenesulfonic, oxalic, palmoic, 2-naphthalenesulfonic, p-toluenesulfonic, cyclohexanesulfamic and saccharic.

This invention also provides a pharmaceutical composition comprising the instant compound and a pharmaceutically acceptable carrier.

Pharmaceutically acceptable carriers are well known to those skilled in the art and include, but are not limited to, from about 0.01 to about 0.1 M and preferably 0.05 M phosphate buffer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a disorder ameliorated by reducing TNF-α production and/or p38 activity in appropriate cells, which comprises administering to the subject a therapeutically effective dose of the instant pharmaceutical composition.

In one embodiment, the disorder is an inflammatory disorder. In another embodiment, the disorder is an AIDS-related disorder. Examples of disorders treatable by the instant pharmaceutical composition include, without limitation, rheumatoid arthritis, osteoporosis, osteoarthritis, allergic inflammation, periodontal disorder, inflammatory bowel disorder, septic shock, insulin-dependent diabetes mellitus, non-insulin-dependent diabetes, cachexia, pulmonary fibrosis, myasthenia gravis, Crohn's disease, hepatitis, primary biliary cirrhosis, acute pancreatitis, allograph rejection, glioblastoma, alopecia areta, psoriasis, ischemia, congestive heart failure, restenosis, atherosclerosis, systemic lupus erythematosus, nephritis, Guillain-Barre Syndrome, viral myocarditis, HIV replication, T-cell depletion in HIV infection, cognitive deficits induced by neuronal inflammation, multiple sclerosis, stroke, neuropathic pain, HIV dementia and Alzheimer's disease. In the preferred embodiment, the disorder is rheumatoid arthritis.

As used herein, the term “subject” includes, without limitation, any animal or artificially modified animal having a disorder ameliorated by reducing TNF-α production and/or p38 activity in appropriate cells. In the preferred embodiment, the subject is a human.

As used herein, “appropriate cells” include, by way of example, cells which secrete or are capable of secreting TNF-α, and cells wherein p38 has been activated. Specific examples of appropriate cells include, without limitation, monocytes, macrophages, T lymphocytes, fibroblasts, dendritic cells, Langerhans cells, Kuppfer cells and astroglial cells.

Administering the instant pharmaceutical composition can be effected or performed using any of the various methods known to those skilled in the art. The instant compounds can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of the instant pharmaceutical composition. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0 μg/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

This invention still further provides a method of preventing an inflammatory response in a subject, comprising administering to the subject a prophylactically effective amount of the instant pharmaceutical composition either preceding or subsequent to an event anticipated to cause the inflammatory response in the subject. In the preferred embodiment, the event is an insect sting or an animal bite.

As used herein, the following chemical terms shall have the meanings as set forth in the following paragraphs: “independently”, when in reference to chemical substituents, shall mean that when more than one substituent exists, the substituents may be the same or different;.

“Alkyl” shall mean straight, cyclic and branched-chain alkyl. Unless otherwise stated, the alkyl group will contain 1-20 carbon atoms. Unless otherwise stated, the alkyl group may be optionally substituted with one or more groups such as halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, carboxamide, hydroxamic acid, sulfonamide, sulfonyl, thiol, aryl, aryl(C₁-C₈)-alkyl, heterocyclyl, and heteroaryl.

“Alkoxy” shall mean —O-alkyl and unless otherwise stated, it will have 1-8 carbon atoms.

“Halogen” or “halo” shall mean fluorine, chlorine, bromine or iodine; “PH” or “Ph” shall mean phenyl; “Ac” shall mean acyl; “Bn” shall mean benzyl; “Me” shall mean methyl.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups include, for example, pyridinyl, pyrazinyl, pyrimidinyl, pyridazinyl, pyrroyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, 2-oxazepinyl, azepinyl, N-oxo-pyridyl, 1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, benzothiopyranyl, indazolyl, indolizinyl, benzofuryl, chromonyl, coumarinyl, cinnolinyl, quinoxalinyl, indazolyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl, and furyl. The heteroaryl group may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, or carboxamide. Heteroaryl may be substituted with a mono-oxo to give for example a 4-oxo-1H-quinoline.

The terms “heterocycle,” “heterocyclic,” and “heterocyclo” refer to an optionally substituted, fully or partially saturated cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom.

Exemplary monocyclic heterocyclic groups include pyrrolidinyl; oxetanyl; pyrazolinyl; imidazolinyl; imidazolidinyl; oxazolyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; and the like. Exemplary bicyclic heterocyclic groups include quinuclidinyl; tetrahydroisoquinolinyl; dihydroisoindolyl; dihydroquinazolinyl (such as 3,4-dihydro4-oxo-quinazolinyl); dihydrobenzofuryl; dihydrobenzothienyl; dihydrobenzothiopyranyl; dihydrobenzothiopyranyl sulfone; dihydrobenzopyranyl; indolinyl; isochromanyl; isoindolinyl; piperonyl; tetrahydroquinolinyl; and the like.

Preferably, heterocycles are selected from the following groups:

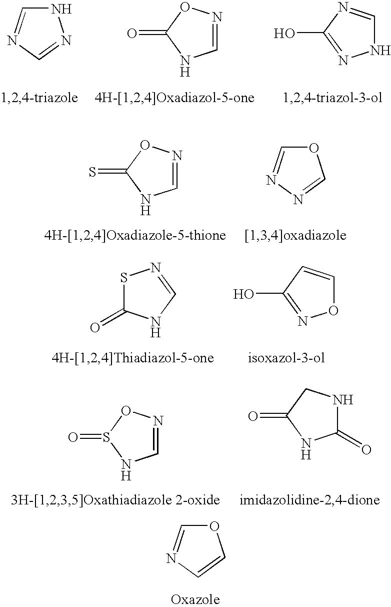

Substituted aryl, substituted heteroaryl, and substituted heterocycle may also be substituted with a second substituted-aryl, a second substituted-heteroaryl, or a second substituted-heterocycle to give, for example, 4-pyrazol-1-yl-phenyl or 4-pyridin-2-yl-phenyl.

Designated numbers of carbon atoms (e.g., C₁-₈) shall refer independently to the number of carbon atoms in an alkyl or cycloalkyl moiety or to the alkyl portion of a larger substituent in which alkyl appears as its prefix root.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their components enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Unless otherwise noted, the following chemical terms shall have the meanings as set forth in this paragraph: “independently”, when in reference to chemical substituents, shall mean that when more than one substituent exists, the substituents may be the same or different; “TCA” shall mean trichloroacetic acid; “FCS” shall mean fetal calf serum; and “RPMI” shall mean the medium from the Roswell Park Memorial Institute (Sigma cat # R0833); “DMF” shall mean N,N-dimethylformamide; “THF” shall mean tetrahydrofuran; and “TBAF” shall mean tetrabutylammonium fluoride.

This invention will be better understood by reference to the Experimental Details which follow, but those skilled in the art will readily appreciate that these are only illustrative of the invention as described more fully in the claims which follow thereafter. Additionally, throughout this application, various publications are cited. The disclosure of these publications is hereby incorporated by reference into this application to describe more fully the state of the art to which this invention pertains.

Experimental Details

Schemes 1-10, wherein
R₇ is selected from hydrogen, optionally substituted alkyl, alkyl-C(O)R′, alkyl-C(NOH)R′, alkyl-C(O)NR′R″, alkyl-NR′R″, CF₂H, CF₃, alkyl-aryl, alkyl-heterocycle, optionally substituted aryl, optionally substituted heteroaryl, and optionally substituted heterocycle, wherein R′ and R″ are independently selected from hydrogen, optionally substituted alkyl, optionally substituted aryl, and optionally substituted heterocycle; and
R₈ is selected from NHNHC(O)R₇, NR′R″
   show the synthesis of Compounds IA through IPP of the present invention.

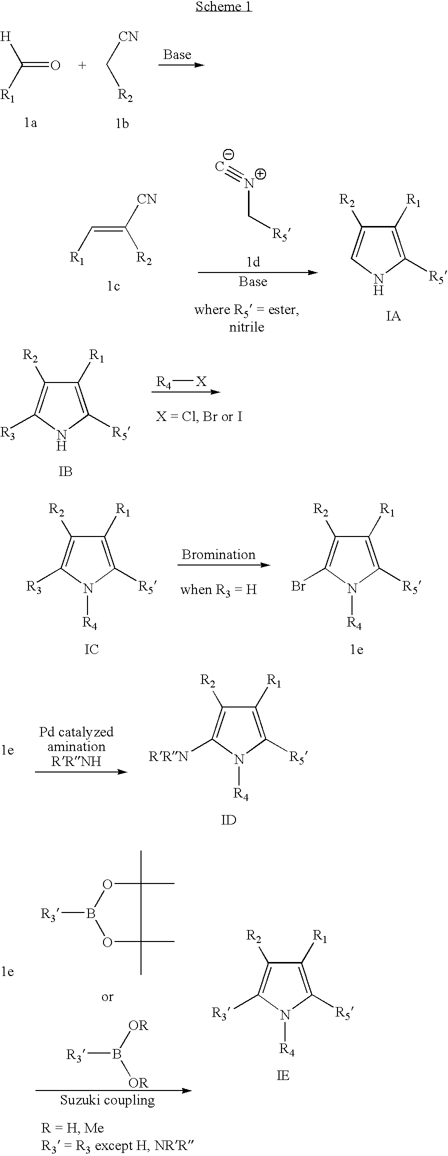

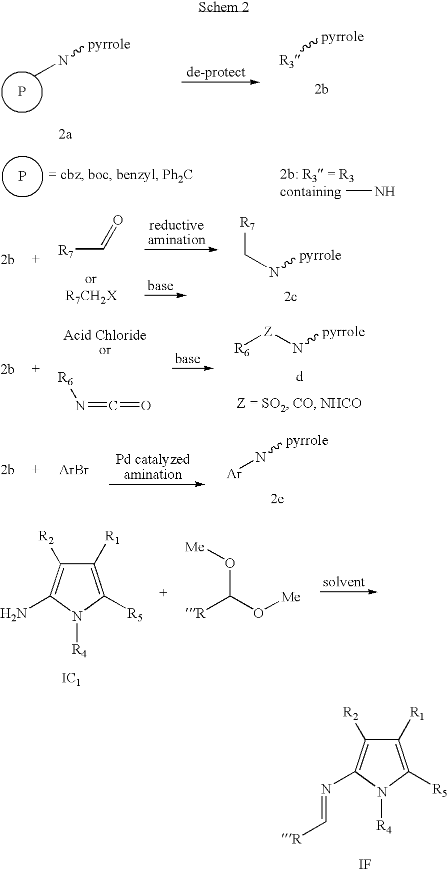

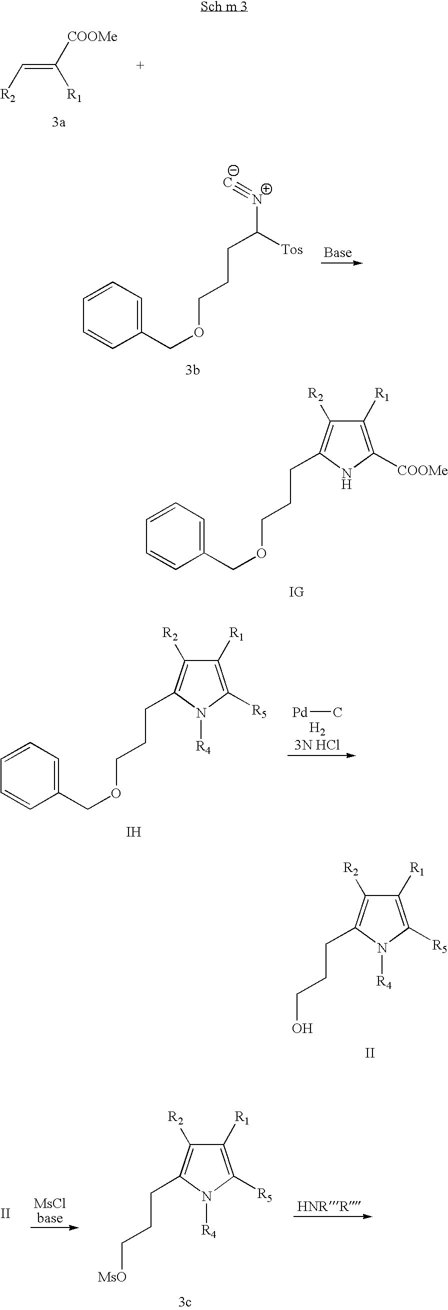

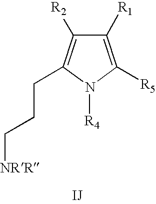

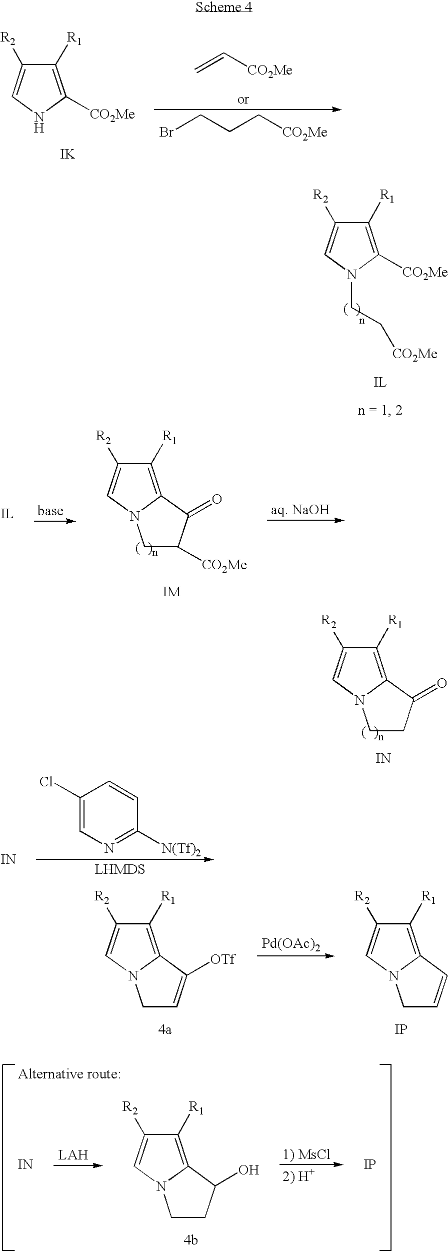

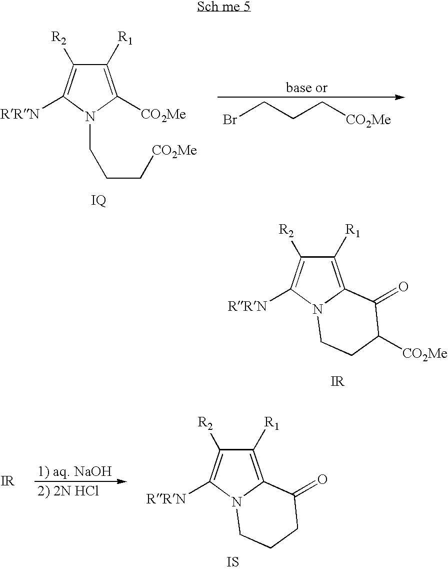

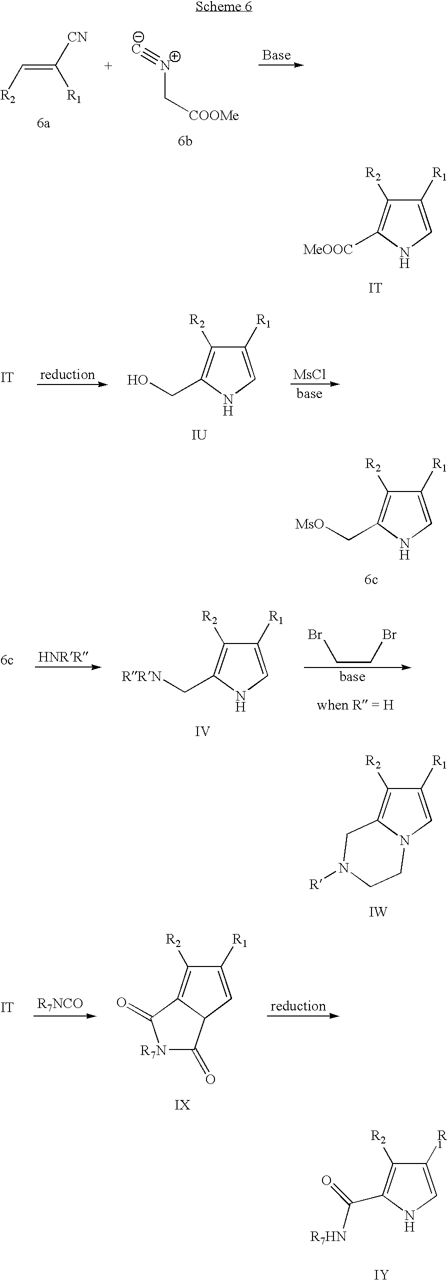

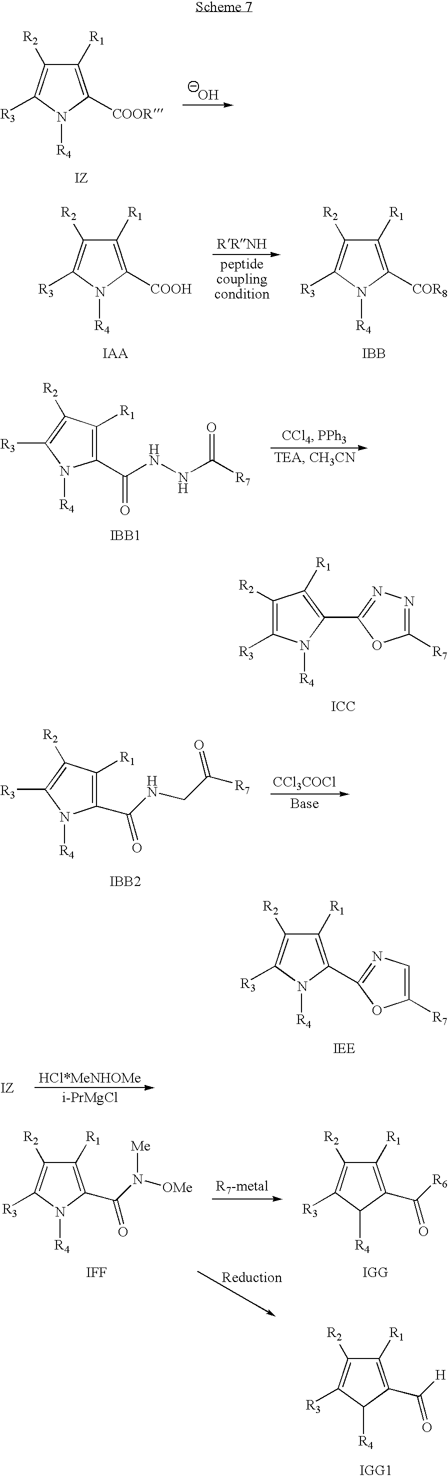

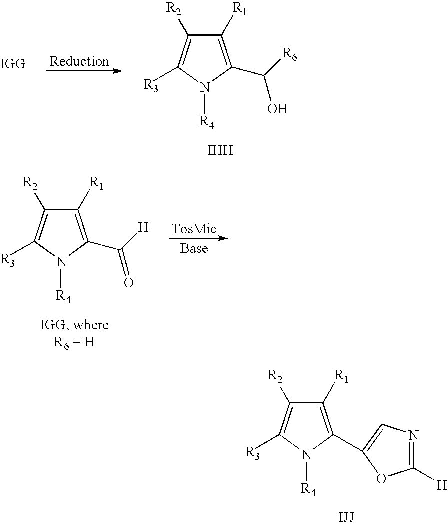

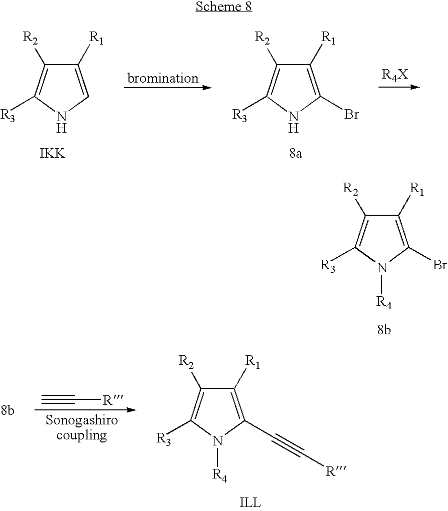

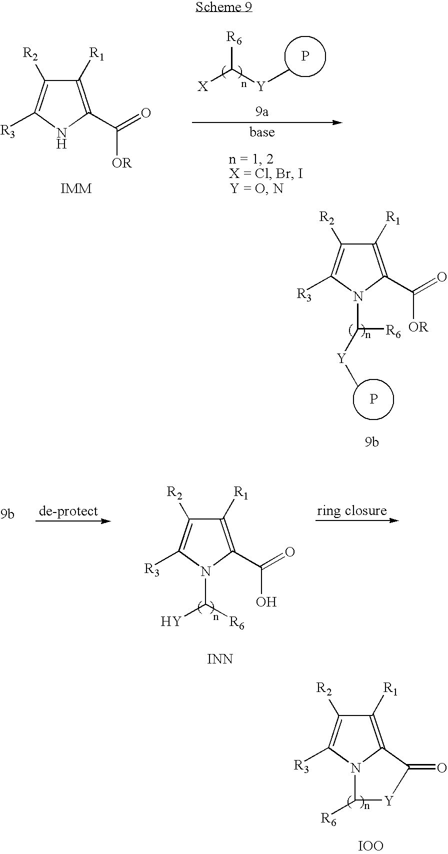

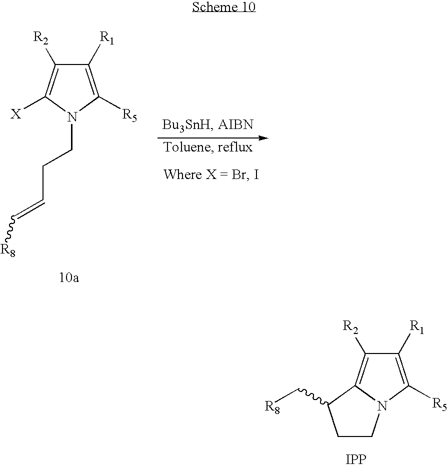

### EXAMPLE 1

### [(4-Fluorophenyl)methylene]-4-pyridineacetonitrile (Compound 1)

4-pyridylacetonitrile hydrochloride (40 g, 0.32 mol), 4-fluorobenzaldehyde (36.3 mL, 0.34 mol) and potassium carbonate (16 g, 0.12 mol) were refluxed in methanol (1800 mL) for 4 hours using a mechanical stirrer. The reaction mixture was cooled in an ice bath and diluted with water (600 mL) while stirring. The resulting precipitate was filtered and washed with water, then allowed to air dry on the vacuum filter for 2 hours to give Compound 1 (56 g, 97%) as a colorless solid: ¹H NMR (CDCl₃) δ 8.65-8.78 (2H, dd), 7.94-8.05 (2H, m), 7.70 (1H, s), 7.55-7.60 (2H, dd), 7.18-7.27 (2H, dd).

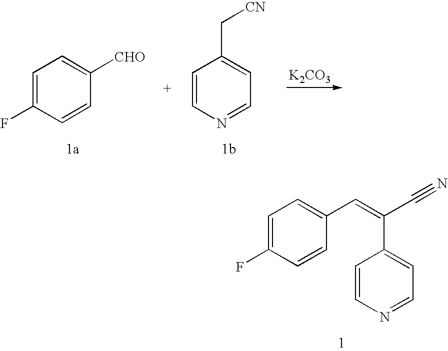

### EXAMPLE 2

### 3-(4-Fluorophenyl )-4-(4-pyridinyl )-1H-pyrrole-2-carboxylic acid methyl ester (Compound 2)

A mixture of Compound 1 (25 g, 0.11 mol) and methyl isocyanoacetate (10.7 mL, 0.11 mol) in tetrahydrofuran (800 mL) was added dropwise to a mixture of potassium-t-butoxide (15 g, 0.13 mol) in tetrahydrofuran (200 mL) at 5° C., at a rate close to 133 mL per hour in 6 hours. (After 3 hrs the reaction mixture turned green then very dark green as the product started to form. MS analysis of the mixture indicated the Michael addition product (M+1=324) until the green color formed then product peak was shown in MS.) After the addition was complete, the mixture was stirred for 1 hour, then diluted with water (1 L) and extracted into ethyl acetate (1×500 mL, 1×200 mL). The organic layers were washed with water (1×300 mL), then brine (200 mL) and dried over sodium sulfate. The drying agent was filtered and the solvent removed in vacuo to give a dark solid. This resulting solid was triturated with methylene chloride and the resulting precipitate was filtered to Compound 2 (11.2 g) as a light yellow solid. The filtrate was purified on silica gel eluting with 60% ethyl acetate in hexane to give another 2.5 g of Compound 2 (total 13.7 g, 42%): ¹H NMR (DMSO) δ 12.45 (NH, s, br), 8.31-8.36 (2H, dd), 7.56-7.60 (1H, d), 7.13-7.28 (4H, m), 6.98-7.00 (2H, dd), 3.65 (3H, s).

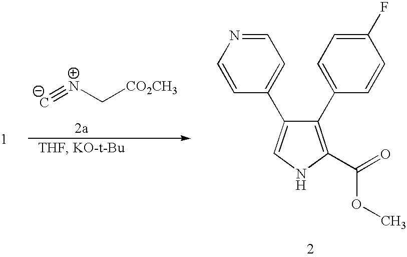

### EXAMPLE 3

### 3-(4-Fluorophenyl)-1-methyl-4-(4-pyridinyl)-1H-pyrrole-2-carboxylic acid methyl ester (Compound 3)

Sodium hydride (60% in mineral oil, 1.7 g) was washed 3 times with hexane and suspended in DMSO (98 mL). Compound 2 (11.2 g, 0.38 mol) was added portionwise while maintaining the temperature at 20° C. using a cool water bath. After addition was complete the reaction mixture was stirred at room temperature for 20 minutes. Next, methyl iodide (2.4 mL, 0.039 mol) was added quickly in one portion. The resulting mixture was stirred for 45 minutes then the excess NaH was carefully quenched with water. The mixture was then diluted to 500 mL with water and stirred for one hour. The precipitating solid was filtered and washed with water, then washed with pet ether and air-dried. After drying, Compound 3 (10.5 g, 90%) was obtained and was pure enough to use for the next reaction: ¹H NMR (DMSO) δ 8.30-8.36 (2H, dd), 7.69 (1H, s), 7.18-7.25 (4H, m), 6.91-7.96 (2H, dd), 3.94 (3H, s), 3.51 (3H, s).

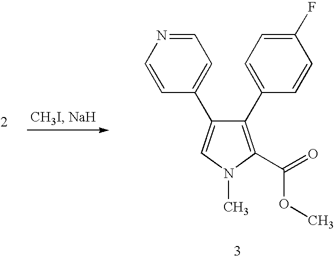

### EXAMPLE 4

### 5-Bromo-3-(4-fluorophenyl)-1-methyl-4-(4-pyridinyl)-1H-pyrrole-2-carboxylic acid methyl ester (Compound 4)

Compound 3 (10.5 g, 0.39 mol) was dissolved in methylene chloride (1 L) and NBS (7.2 g, 0.40 mol) was added portionwise with stirring. After 2 hours the TLC (ether) and MS showed that the reaction was complete. The reaction mixture was washed with dilute sodium bicarbonate solution (300 mL) and brine (200 mL). The organic layer was dried over sodium sulfate and filtered. The organic solution was then poured onto a bed of silica gel (500 mL) and eluted with methylene chloride. Next the silica was eluted with ether to collect a yellow solution that contained the desired product. Evaporation of the solvent gave a yellow solid, which was triturated with pet ether and filtered to give Compound 4 (6.4 g, 48%): ¹H NMR (CDCl₃) δ 8.42-8.50 (2H, dd), 6.90-7.09 (6H, m), 4.08 (3H, s), 3.60 (3H, s).

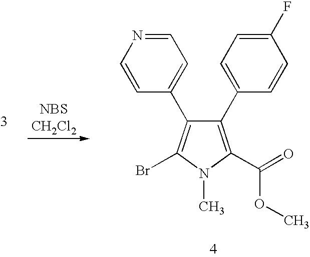

### EXAMPLE 5

### 3-(4-Fluorophenyl)-1-methyl-5-[2-(4-morpholinyl)ethyl]-4-(4-pyridinyl)-1H-pyrrole-2-carboxylic acid methyl ester (Compound 5)

A mixture of BINAP (0.345 g, 0.554 mmol), Pd₂(dba)₃ (0.018 g, 0.0196 mmol) and toluene (0.5 mL) was added to an oven-dried, nitrogen purged test tube (16×100 mm) equipped with a Teflon stir bar. This mixture was heated to 100° C. and stirred for 30 minutes. Next, cesium carbonate (0.351 g, 1.08 mmol), Compound 4 (0.300 g, 0.77 mmol) and 4-(2-aminoethyl)morpholine (0.120 g, 0.92 mmol) were added and the test tube was washed with additional toluene (0.5 mL). The resulting mixture was stirred vigorously under nitrogen at 100° C. MS analysis indicated the reaction completed after 24 hr. The reaction mixture was then cooled to room temperature and 0.5 mL of water was added followed by 5 mL of methanol. The test tube was then washed with excess methanol and the mixture was filtered through Celite. The filtrate was extracted with ethyl acetate and dried over sodium sulfate. The organic layers were evaporated under vacuum and the resulting residue was dissolved in methylene chloride and purified on a silica gel 60 column eluting with 10% MeOH in ethyl acetate to give Compound 5 (0.195 g, 65%): ¹H NMR (CDCl₃) δ 8.36-8.41 (2H, dd), 7.09-7.21 (2H, m), 6.88-6.95 (4H, m), 3.85 (3H, s), 3.57 (3H, s), 3.50-3.56 (4H, m), 2.93-3.01 (2H, m), 2.39-2.45 (2H, m), 2.50-2.30 (4H, m).

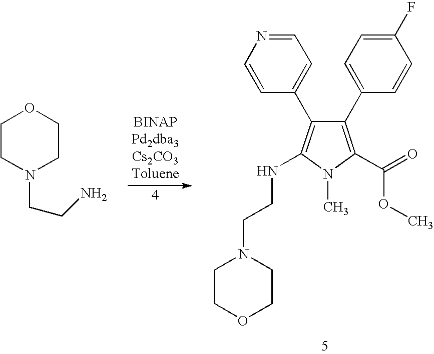

### EXAMPLE 6

### 4-[4-(4-Fluorophenyl)-5-(methoxycarbonyl)-1-methyl-3-(4-pyridinyl)-1H-pyrrol-2-yl]-3,6-dihydro-1(2H)-pyridinecarboxylic acid phenylmethyl ester (Compound 6)

Compound 4 (208 mg, 0.53 mmol), Compound 6a (194 mg, 0.565 mmol; prepared according to the procedure described by Eastwood, P. R. Tetrahedron Lett. 2000, 41(19), 3705-3708), Pd₂(dba)₃ (0.013 g, 0.0142 mmol) and tri-p-tolyl-phosphine (28 mg, 0.092 mmol) were added to an oven-dried test tube. Next the test tube was diluted with toluene (0.6 mL), 1M sodium carbonate (0.3 mL) and ethanol (0.3 mL). This mixture was heated to 80° C. for 20 hours under nitrogen. Upon cooling, the mixture was diluted with water (35 mL) and extracted into methylene chloride. The organic layer was washed with water 3 times and once with brine, dried over sodium sulfate and evaporated to an oil. Purification on silica gel eluting with 60% ethyl acetate in hexanes gave Compound 6 (130 mg, 46%). MS (M+1, 526)

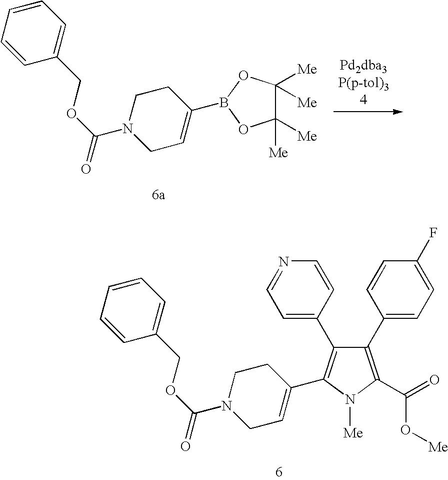

### EXAMPLE 7

### 3-(4-Fluorophenyl)-1-methyl-4-(4-pyridinyl)-5-(1,2,3,6-tetrahydro-4-pyridinyl)-1H-pyrrole-2-carboxylic acid methyl ester (Compound 7)

Compound 6 (120 mg, 0.228 mmol) was dissolved in ethanol (100 mL) containing concentrated hydrochloric acid (0.5 mL) and reduced under a hydrogen atmosphere with 10% Pd-C (20 mg) using a Parr hydrogenator. After 24 hours the catalyst was filtered and the resulting filtrate was treated with triethylamine. The solvent was evaporated and the residue was dissolved in ethyl acetate. This solution was washed with water 3 times, then once with brine and dried over sodium sulfate. Removal of the solvent in vacuo gave Compound 7 (80 mg, 90%) as an oil. MS (M+1, 392)

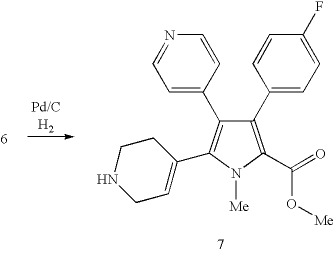

### EXAMPLE 8

### 3-(4-Fluorophenyl )-1-methyl-5-(1-piperazinyl)-4-(4-pyridinyl)-1H-pyrrole-2-carboxylic acid methyl ester (Compound 8)

Compound 8a (Example 6 synthesis) (440 mg, 0.890 mmol) was dissolved in methylene chloride (200 mL) containing trifluoroacetic acid (3 mL) and heated to reflux for 16 hours. The reaction mixture was cooled and evaporated in vacuo to give an oil. The oil was dissolved in ether and washed with saturated sodium bicarbonate solution (2 times), then with brine. The organic layers were dried over sodium sulfate and treated dropwise with 1N hydrochloric acid. The resulting precipitate was filtered and washed with ether. The solid was dried under vacuum at 60° C. to give the dihydrochloride salt of Compound 8 (334 mg, 80%).

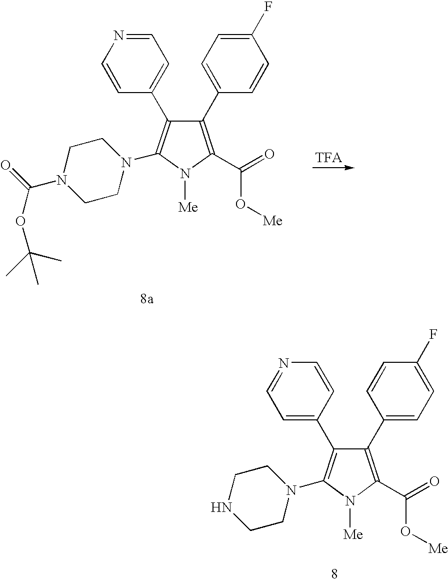

### EXAMPLE 9

### 5-Amino-3-(4-fluorophenyl )-1-methyl-4-(4-pyridinyl )-1H-pyrrole-2-carboxylic acid methyl ester (Compound 9)

A mixture of Compound 9a (Example 5 synthesis) (0.350 g, 0.715 mmol), 2.9 equivalents of sodium acetate (0.170 g, 2.07 mmol) and 1.8 equivalents of hydroxylamine hydrochloride (0.0894 g, 1.287 mmol) was stirred in methanol (10 mL) in nitrogen atmosphere. The reaction mixture was then refluxed for 24 hours, cooled, diluted with ethyl acetate (250 mL) and the organic layer was washed with 0.1N NaOH (1×100 mL) and dried over sodium sulfate. The drying agent was filtered and the solvent removed in vacuo to give a white solid. The solid was dissolved in methylene chloride and purified on silica gel eluting with 50% ethyl acetate in hexanes to afford Compound 9 (0.220 g, 94.5%) as a white solid. MS (M+1, 326)

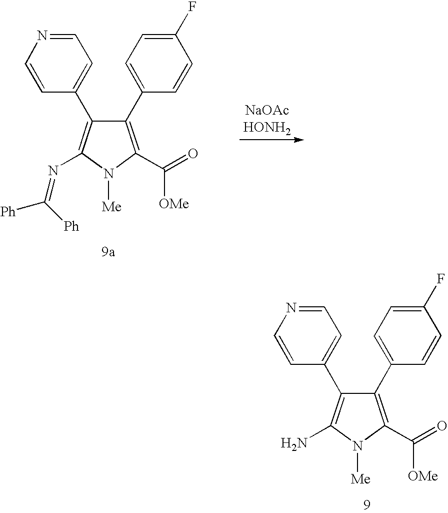

### EXAMPLE 10

### 3-(4-Fluorophenyl)-1-methyl-5-(4-methyl-1-piperazinyl)-4-(4-pyridinyl)-1H-pyrrole-2-carboxylic acid methyl ester (Compound 10)

Sodium triacetoxyborohydride (250 mg, 1.17 mmol) was added portionwise to a rapidly stirring solution of Compound 8 (115 mg, 0.292 mmol) and 37% formaldehyde in water (25 mg) in dichloroethane (2 mL). After 1 hour the reaction was complete as determined by MS and TLC. The reaction mixture was quenched with 2N sodium hydroxide (0.5 mL). The product was extracted into ethyl acetate and washed with brine. After drying over sodium sulfate the solvent was removed under vacuum to give Compound 10 (97 mg) as an oil which was pure by NMR. This oil was dissolved in ether and 1N HCl in ether was added dropwise and the resulting precipitate was filtered, washed with ether and dried under vacuum at 60° C. to give dihydrochloride salt of Compound 10 (56 mg). MS (M+1, 409)

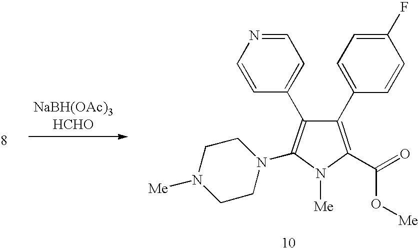

### EXAMPLE 11

### 3-(4-Fluorophenyl)-1-methyl-5-[methyl[2-(4-morpholinyl)ethyl]amino]-4-(4-pyridinyl)-1H-pyrrole-2-carboxylic acid methyl ester (Compound 11)

Sodium hydride (60% in mineral oil, 0.0426 g) was washed 3 times with hexanes and suspended in DMSO (15 mL) and cooled to 5° C. Compound 5 (0.390 g, 0.89 mmol) was added portionwise and after the addition was complete the stirred mixture was allowed to warm to room temperature. Methyl Iodide (0.128 g, 0.907 mmol) was then added quickly in one portion. The reaction was monitored by MS and TLC (eluting with 5% MeOH in ethyl acetate). After an hour, water (5 mL) was carefully added and the reaction mixture was extracted with ethyl acetate. The extracts were washed with brine 3 times then dried over sodium sulfate and the solvent removed in vacuo. The product was purified on silica gel 60 eluting with 5% methanol in ethyl acetate to give Compound 11 (0.994 g, 51%): ¹H NMR (CDCl₃) [symbol] 8.38-8.42 (2H, dd), 7.05-7.10 (2H, m), 6.89-6.95 (4H, m), 3.85 (3H, s), 3.67-3.69 (4H, m), 3.57 (3H, s), 2.89-2.93 (2H, m), 2.88 (3H, s), 2.33-2.42 (2H, m), 2.32-2.50 (4H, m).

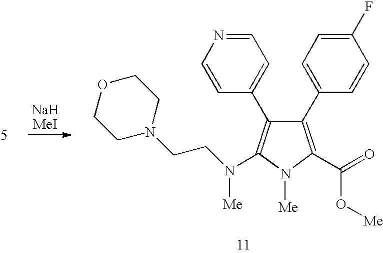

### EXAMPLE 12

### 3-(4-Fluorophenyl)-1-methyl-5-[4-(methylsulfonyl)-1-piperazinyl]-4-(4-pyridinyl)-1H-pyrrole-2-carboxylic acid methyl ester (Compound 12)

A solution of methane sulfonylchloride (20 μL, 29 mg, 0.253 mmol) in methylene chloride (5 mL) was added dropwise to a stirred solution of Compound 8 (100 mg, 0.254 mmol) and triethylamine (0.5 mL) in methylene chloride (50 mL). The mixture was stirred for 4 hours then water was added and the methylene chloride layer was diluted to 100 mL. The organic layer was washed with water, then brine and dried over sodium sulfate. Removal of the solvent in vacuo gave an oil which was purified on a preparative TLC plate eluting with 60% ethyl acetate in hexanes to provide Compound 12 (30 mg, 25%). MS (M+1, 473)

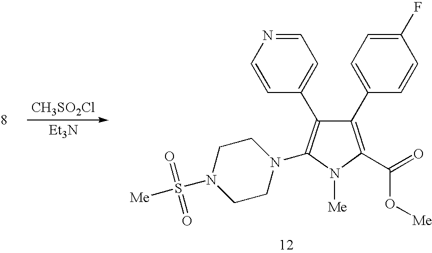

### EXAMPLE 13

### 5-[4-[(Ethylamino)carbonyl]-1-piperazinyl]-3-(4-fluorophenyl)-1-methyl-4-(4-pyridinyl)-1H-pyrrole-2-carboxylic acid methyl ester (Compound 13)

Sodium hydride (60% in mineral oil, 12 mg) was washed 3 times with hexanes and suspended in THF (10 mL). Compound 8 (100 mg, 0.254 mmol) was added portionwise and the mixture was stirred at 25° C. for 30 minutes. Ethyl isocyanate (18 mg, 0.253 mmol) was added and the resulting mixture was stirred for 4 hours. The reaction mixture was then quenched with water and extracted into ethyl acetate. The organic layers were washed with water then brine. After drying over sodium sulfate, the solvent was removed and the residue was purified on a preparative TLC plate eluting with 60% ethyl acetate in hexanes to give Compound 13 (83 mg) as an oil. This oil was dissolved in ether and 1N HCl in ether was added dropwise and the resulting precipitate was filtered, washed with ether and dried under vacuum at 60° C. to give the hydrochloride salt of Compound 13 (75 mg). MS (M+1, 466)

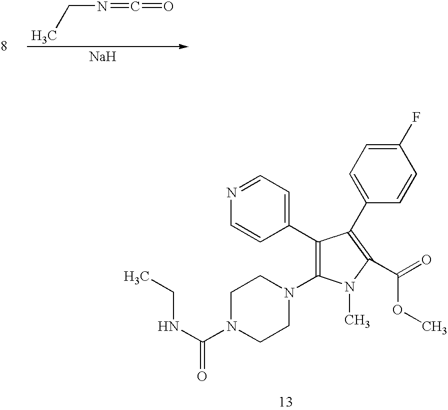

### EXAMPLE 14

### 5-[4-(3-Acetylphenyl)-1-piperazinyl]-3-(4-fluorophenyl)-1-methyl-4-(4-pyridinyl)-1H-pyrrole-2-carboxylic acid methyl ester (Compound 14)

Pd₂(dba)₃ (0.005 g, 0.025 eq.) and BINAP (0.009 g, 0.070 eq.) were added to a tube purged with nitrogen. The tube was again purged with nitrogen and 3-bromoacetophenone (0.028 mL, 1.0 eq.), Compound 8 (0.100 g, 1.20 eq.), Cs₂CO₃ (0.094 g, 1.40 eq.) and toluene (1 mL) were sequentially added to the tube. The mixture was heated to reflux for 6 days. Upon cooling to room temperature, the mixture was diluted with ether then filtered through Celite to remove the insolubles and extracted into ethyl acetate. The organic extract was washed with water, dried over sodium sulfate and concentrated in vacuo. The resulting residue was purified by column chromatography on silica gel eluting with a gradient of 10%-50% ethyl acetate in hexanes to give Compound 14 (40.4 mg) as a white solid. Compound 14 was dissolved in ether and a solution of 1N HCl in ether was added dropwise until a precipitate formed. The precipitate was concentrated in vacuo followed by column chromatography eluting with ethyl acetate to give the HCl salt of Compound 14 (16.5 mg).

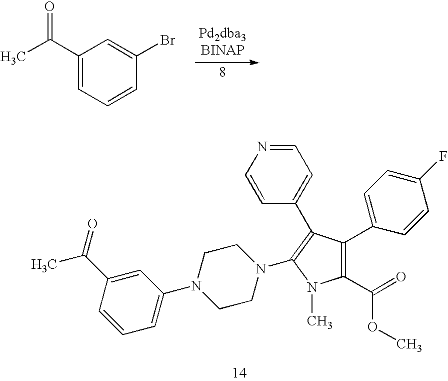

### EXAMPLE 15

### 5-[[(Dimethylamino)methylidene]amino]-3-(4-fluorophenyl)-1-methyl-4-(4-pyridinyl)-1H-pyrrole-2-carboxylic acid methyl ester (Compound 15)

5-Amino-3-(4-fluoro-phenyl)-1-methyl-4-pyridin-4-yl-1H-pyrrole-2-carboxylic acid methyl ester (50 mg, 0.15 mmol) was stirred in DMF (0.12 mL). Dimethylformamide dimethylacetal (0.12 mL, 0.92 mmol) was added and the mixture stirred at room temperature for 16 hours. The solvent was concentrated in vacuo and the resulting residue was dissolved in methylene chloride and washed with water. The solution was dried over MgSO₄ and concentrated in vacuo. The residue was crystallized by trituration with hexanes. The crystals were dried under high vacuum to obtain Compound 15 (0.0421 g). MS (M+1, 381)

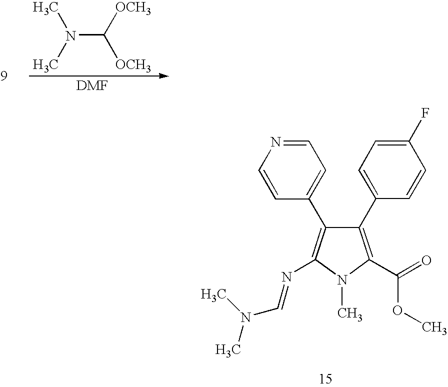

### EXAMPLE 16

### 3-(4-Fluorophenyl)-5-[3-(phenylmethoxy)propyl]-4-(4-pyridinyl)-1H-pyrrole-2-carboxylic acid ethyl ester (Compound 16)

1-(4-Tolylsulfonyl )-1-(3-benzyloxy-propyl)methyl isocyanide Compound 16a (29.84 g, 0.0869 mol) and ethyl 4-fluoro-□-[(4-pyridyl)methylene]benzeneacetic acid Compound 16b (23.5 g, 0.0869 mol) were dissolved in dry THF (200 mL) and added dropwise to a cooled (0° C.) mixture of potassium-t-butoxide (9.8 g, 0.0869 mol) in dry THF (400 mL). After the addition was complete, the mixture was stirred for 1 hour then poured into H₂O (1400 mL) and extracted into ethyl acetate (2×500 mL). The organic layers were washed with water (100 mL), dried over sodium sulfate and evaporated in vacuo to give an oil. Trituration with acetonitrile gave an impure solid, which was purified on silica gel eluting with 70% ethyl acetate in hexanes. Recrystallization from ethyl acetate gave Compound 16 (1.9 g). MS (M+1,473)

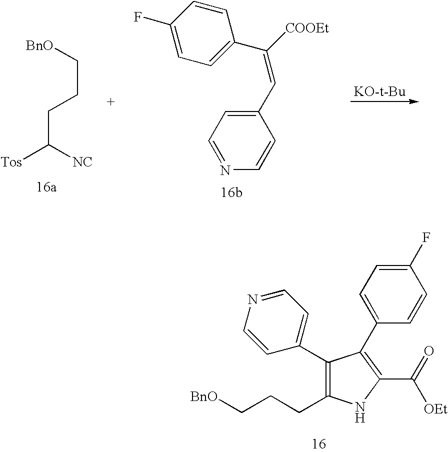

### EXAMPLE 17

### 3-(4-Fluorophenyl)-5-(3-hydroxypropyl)-1-methyl-4-(4-pyridinyl)-1H-pyrrole-2-carboxylic acid ethyl ester (Compound 17)

A solution of Compound 16 (1.55 g, 0.0033 mol) in ethanol (125 mL) containing concentrated HCl (0.3 mL) was added to 10% Pd on carbon (0.2 g). This mixture was placed in a hydrogen atmosphere for 16 hours on a Parr hydrogenator at 50 PSI. The mixture was filtered through Celite and triethylamine (1.0 mL) was added to the resulting solution, followed by evaporation in vacuo to give a solid. The solid was extracted into ethyl acetate (100 mL) and washed with water (3×50 mL). The organic layers were dried over sodium sulfate and concentrated in vacuo to give Compound 17 (0.75 g, 60%) as a white solid: MS (M+1, 383)

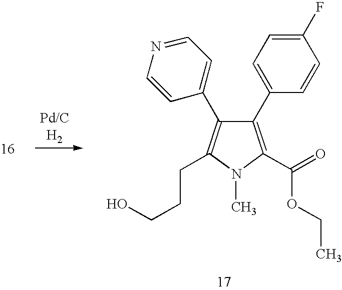

### EXAMPLE 18

### 3-(4-fluorophenyl)-1-methyl-5-[3-[(methylsulfonyl)oxy]propyl]-4-(4-pyridinyl)-1H-pyrrole-2-carboxylic acid ethyl ester (Compound 18)

Compound 17 (0.7 g, 0.0018 mol) was combined with triethylamine (0.52 mL, 0.0037 mol) in methylene chloride (50 mL) and cooled to 10° C. Methanesulfonylchloride (0.16 mL, 0.0020 mol) was added dropwise and the resulting mixture was allowed to warm to room temperature. The mixture was diluted with methylene chloride (50 mL) and washed with water (30 mL). The organic layers were dried over sodium sulfate and evaporated in vacuo to give an oil. This oil was dissolved in ethyl acetate and purified through a bed of SiO₂ (˜20 mL) eluting with ethyl acetate. Evaporation of the solvent in vacuo gave Compound 18 (0.73 g, 87%) as an oil. MS (M+1, 375)

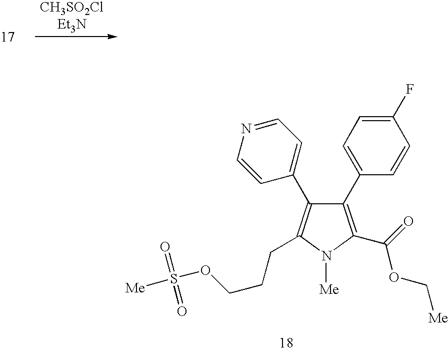

### EXAMPLE 19

### 3-(4-Fluorophenyl)-1-methyl-5-[3-(4-morpholinyl)propyl]-4-(4-pyridinyl)-1H-pyrrole-2-carboxylic acid ethyl ester (Compound 19)

A mixture of Compound 18 (0.15 g, 0.326 mmol), morpholine (0.5 mL) and methylene chloride (25 mL) was refluxed for 16 hours. The mixture was cooled and diluted with methylene chloride (˜100 mL), then washed with water (3×50 mL). The organic layers were dried over sodium sulfate and evaporated in vacuo to give Compound 19 as an oil. This oil was dissolved in ether and 1N HCl in ether was added dropwise and the resulting precipitate was filtered, washed with ether and dried under vacuum at 60° C. to give the di-hydrochloride salt of Compound 19 (100 mg). MS (M+1, 452)

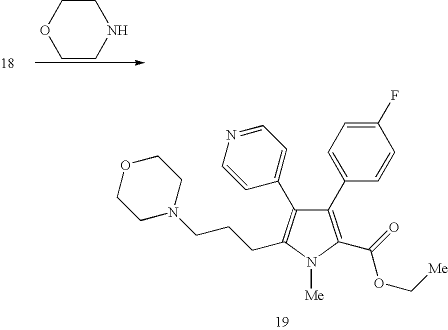

### EXAMPLE 20

### 3-(4-fluorophenyl)-2-(methoxycarbonyl)-4-(4-pyridinyl)-1H-pyrrole-1-propanoic acid methyl ester (Compound 20)

Compound 2 (0.57 g, 1.93 mmol) was refluxed with a catalytic amount of potassium-t-butoxide (21 mg) in methyl acrylate (6 mL) under N₂. The reaction was monitored by TLC. After 1 hr, the reaction mixture was cooled to room temperature and the excess methyl acrylate was removed in vacuo. The residue was taken up in water (15 mL) and ethyl acetate (20 mL). The aqueous layer was extracted with ethyl acetate (2×15 mL). The combined organic layers were washed with water, brine and dried over anhydrous potassium carbonate. Filtration through Celite and concentration gave the crude material. Purification by chromatography (silica gel 60, 55% ethyl acetate/hexanes) yielded Compound 20 (0.707 g, 96%): MS (M+1, 383)

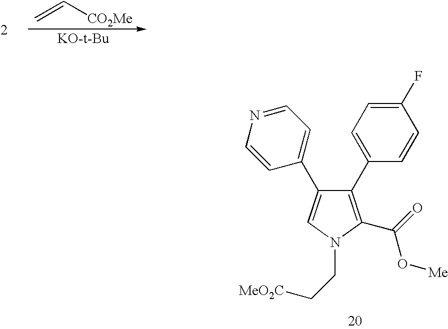

### EXAMPLE 21

### 7-(4-Fluorophenyl)-2,3-dihydro-1-oxo-6-(4-pyridinyl)-1H-pyrrolizine-2-carboxylic acid methyl ester (Compound 21)

Compound 20 (0.5 g, 1.3 mmol) was mixed with dry toluene (20 mL) and sodium methoxide (25% wt in methanol, 1.05 eq.) was added dropwise. The reaction mixture was heated to 80° C. under N₂ and monitored by TLC. After 1.5 hr, the reaction was completed. The mixture was cooled to room temperature and diluted with aq. NH₄Cl and ethyl acetate. The aqueous layer was extracted with ethyl acetate (3×20 mL). The combined organic layers were washed with water, brine and dried over anhydrous sodium sulfate. The resulting solution was filtered through Celite, concentrated in vacuo and the residue was purified by chromatography (silica gel, 100% ethyl acetate) to yield Compound 21 (0.237 g, 52%). MS (M+1, 351)

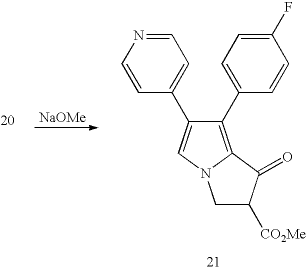

### EXAMPLE 22

### 7-(4-Fluorophenyl)-2,3-dihydro-6-(4-pyridinyl)-1H-pyrrolizin-1-one (Compound

Compound 21 (0.237 g, 0.677 mmol) was refluxed with sodium hydroxide (54.2 mg) in water (15 mL) for 3 hr and then cooled to rt. The mixture was neutralized with dilute acetic acid until pH value is close to 7. The aqueous mixture was extracted with ethyl acetate (3×20 mL). The combined organics were washed with water, brine and dried over anhydrous sodium sulfate. The solution was then filtered through silica gel-Celite pad and concentrated in vacuo to give Compound 22 (0.194 g, 98%): MS (M+1, 293)

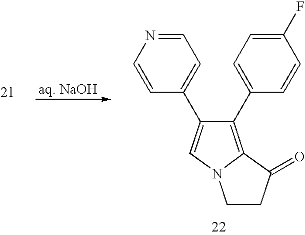

### EXAMPLE 23

### 1-(4-Fluorophenyl)-5,6,7,8-tetrahydro-3-[[2-(4-morpholinyl)ethyl]amino]-8-oxo-2-(4-pyridinyl)-7-indolizinecarboxylic acid methyl ester (Compound 23)

Compound 23a (0.25 g, 0.476 mmol) (Example 5 synthesis) and potassium-t-butoxide (0.059 g, 0.5 mmol) were dissolved in anhydrous THF (5 mL) and the resulting mixture was refluxed for 1 hr under N₂. The mixture was then cooled to room temperature, quenched with aq. NH₄Cl and diluted with ethyl acetate. The aqueous portion was extracted with ethyl acetate (2×20 mL). The combined organic layers were washed with water, brine, dried (anhydrous sodium sulfate), filtered through Celite and concentration in vacuo gave the crude product. The crude material was purified by chromatography (silica gel, 8% methanol/ethyl acetate) to yield Compound 23 (0.14 g, 60%): MS (M+1, 493)

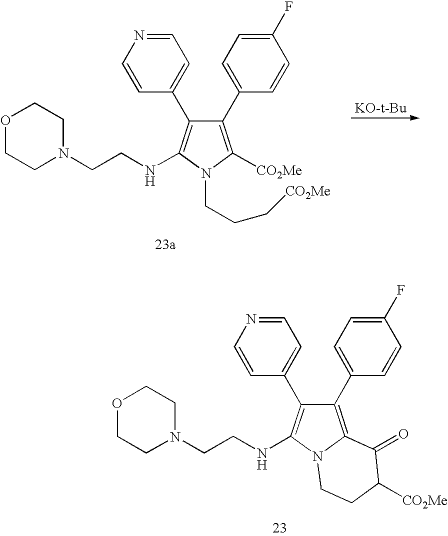

### EXAMPLE 24

### 1-(4-Fluorophenyl)-6,7-dihydro-3-[[2-(4-morpholinyl)ethyl]amino]-2-(4-pyridinyl)-8(5H)-indolizinone (Compound 24)

Compound 23 (89 mg, 0.18 mmol) was refluxed with sodium hydroxide (29 mg) in water (3 mL) for 3 hr and then cooled to room temperature. 6N HCl solution (1 mL) was carefully added and the resulting mixture was refluxed for 0.5 hr. After the reaction was completed and cooled to room temperature, 10% of aqueous sodium hydroxide solution was added to adjust the pH value to 7-8. The aqueous phase was then extracted with ethyl acetate (3×15 mL). The combined organic layers were washed with water, brine, dried over anhydrous sodium sulfate and filtered through Celite. Concentration in vacuo and purification by column chromatography (10% methanol/ethyl acetate) gave Compound 24 (77 mg, 99%): MS (M+1, 435)

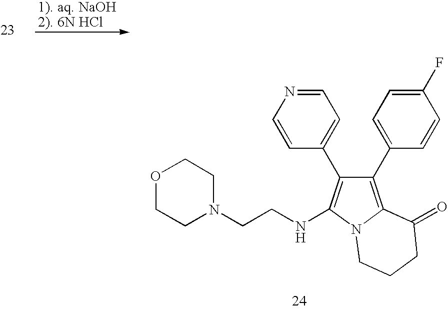

### EXAMPLE 25

### 4-(4-Fluorophenyl )-3-(4-pyridinyl)-1H-pyrrole-2-carboxylic acid methyl ester (Compound 25)

Compound 25a (25 g, 0.11 mol) and methyl isocyanoacetate (Compound 2a, 10.67 mL, 0.11 mol) were dissolved in dry tetrahydrofuran (800 mL) and added dropwise to a solution of potassium-t-butoxide (15 g, 0.13 mol) in dry tetrahydrofuran (200 mL) at 0° C. under nitrogen. The drip rate was kept at ˜133 mL per hour and the addition was completed in 6 hours. Upon completion the reaction mixture was stirred for another hour then diluted with distilled water. The resulting precipitate was filtered under vacuum and air-dried to give Compound 25 (26.4 g, 80%) as a white solid: MS (M+1, 297)

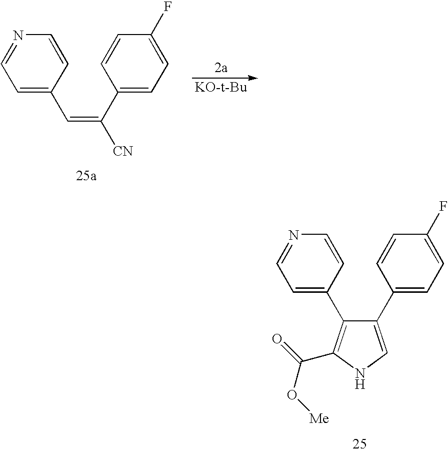

### EXAMPLE 26

### 4-(4-Fluorophenyl)-3-(4-pyridinyl)-1H-pyrrole-2-methanol (Compound 26)

Lithium aluminum hydride (1.0 M in THF, 11 mL, 0.036 mol) was added portionwise under nitrogen atmosphere to a solution of Compound 25 (11.4 g, 0.036 mol) in tetrahydrofuran (400 mL). The reaction was complete after 4 hours. Upon completion the reaction mixture was quenched with H₂O (5.0 mL), 1.0 M NaOH (4.0 mL) and H₂O (5.0 mL) successively. The mixture was filtered through Celite and evaporated under vacuum to give a tan solid. The solid was dissolved in methylene chloride and purified on silica gel eluting with 50% ethyl acetate in hexanes to afford Compound 26 (8.94 g, 90%) as a light tan solid: MS (M+1, 269)

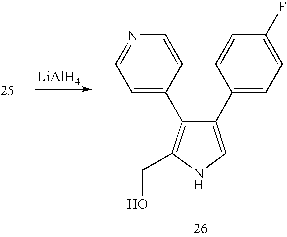

### EXAMPLE 27

### 4-(4-Fluorophenyl)-3-(4-pyridinyl)-1H-pyrrole-2-methanol methanesulfonate (Compound 27)

Methanesulfonyl chloride (2.56 mL, 0.0223 mol) was added at room temperature to a stirred solution of Compound 26 (6.0 g, 0.0223 mol) in pyridine (25 mL) while under nitrogen. The reaction mixture was concentrated under a stream of nitrogen to give Compound 27 (9.25 g) as an a dry black solid which was used without further purification. MS (M+1=251).

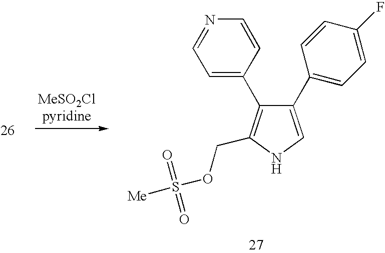

### EXAMPLE 28

### 4-(4-Fluorophenyl)-N-(phenylmethyl)-3-(4-pyridinyl)-1H-pyrrole-2-methanamine (Compound 28)

A 10-fold excess of benzylamine (2.0 mL) was added at room temperature to a stirred solution of Compound 27 (0.50 g) in pyridine (10 mL) while under nitrogen. The reaction mixture was concentrated under a stream of nitrogen for 24 hours to give a black oil. MS (M+1=358). The oil was dissolved in methylene chloride and purified on silica gel eluting with 50% ethyl acetate in hexanes to afford Compound 28 (0.340 g) as a light tan solid: MS (M+1, 358)

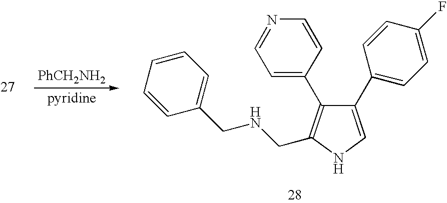

### EXAMPLE 29

### 7-(4-Fluorophenyl)-1,2,3,4-tetrahydro-2-(phenylmethyl)-8-(4-pyridinyl)-pyrrolo[1,2-a]pyrazine (Compound 29)

Compound 28 (0.500 g, 0.0014 mol) was dissolved in DMSO (50 mL) and the solution was chilled to 5° C. Sodium hydride (0.033 g, 0.0014 mol) was added portionwise under nitrogen. The reaction mixture was warmed to room temperature and 1,2-dibromoethane (0.036 mL, 0.0014 mol) was added quickly in one portion. The resulting mixture was stirred for 1 hour then diluted with of water (5 mL) and extracted into ethyl acetate (1×250 mL). The organic layers were washed with brine (3×100 mL), then water (1×100 mL) and dried over sodium sulfate. The drying agent was filtered and the solvent removed in vacuo to give a tan solid. The resulting solid was dissolved in methylene chloride and purified on silica gel eluting with 10% methanol in ethyl acetate affording Compound 29 (0.230 g) as a white solid: MS (M+1, 384)

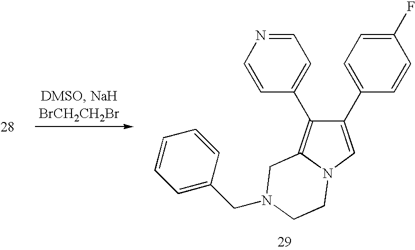

### EXAMPLE 30

### 7-(4-Fluorophenyl)-1,2,3,4-tetrahydro-8-(4-pyridinyl)-pyrrolo[1,2-a]pyrazine (Compound 30)

Palladium hydroxide [20 wt % Pd on carbon, wet (Pearlman's catalyst)] (0.023 g, 10% by weight of starting compound), Compound 29 (0.230 g, 0.599 mmol) and concentrated HCl (1.0 mL) were dissolved in methanol (100 mL) in a Parr hydrogenation bottle. The reaction vessel was filled with H₂ (50 psi) and shaken for 24 hours at room temperature. The reaction mixture was filtered through Celite to remove the Pd and washed with CH₂Cl₂. The organic layers were dried over sodium sulfate and concentrated in vacuo to give Compound 30 (0.080 g, 97%) as a light yellow solid: MS (M+1, 294)

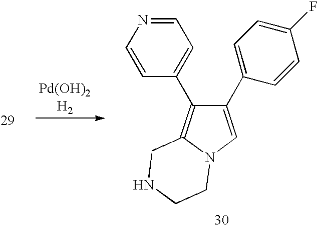

### EXAMPLE 31

### 2-Ethyl-6-(4-fluorophenyl)-7-(4-pyridinyl)-1H-pyrrolo[1,2-c]imidazole-1,3(2H)-dione (Compound 31)

Triethylamine (0.123 mL, 0.00168 mol) and ethyl isocyanate (0.207 mL, 0.00168 mol) were added to a solution of Compound 25 (0.50 g, 0.00168 mol) in methylene chloride (100 mL) and the mixture was refluxed for 5 h under nitrogen. The reaction mixture was allowed to cool to room temperature and diluted with ethyl acetate (150 mL). The organic layers were dried over sodium sulfate and concentrated in vacuo to give a yellow oil. The resulting oil was dissolved in methylene chloride and purified on silica gel eluting with 10% methanol in ethyl acetate to afford Compound 31 (0.50 g, 74%) as a light tan solid: MS (M+1, 336)

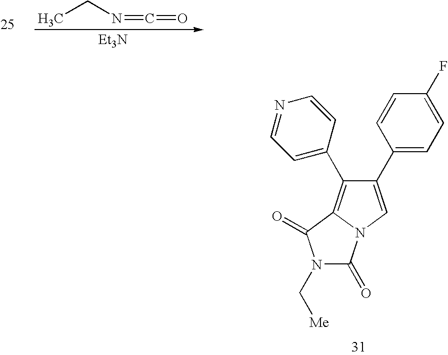

### EXAMPLE 32

### N-Ethyl-4-(4-fluorophenyl)-3-(4-pyridinyl)-1H-pyrrole-2-carboxamide (Compound 32)

Sodium borohydride (0.10 g, 0.00252 mol) was added at room temperature to a solution of Compound 31 (0.50 g, 0.00125 mol) in ethanol (200 mL). The mixture was stirred under nitrogen for 4 hours then diluted with water (10 mL) and extracted into ethyl acetate (250 mL). The organic layers were dried over sodium sulfate, filtered and the solvent removed in vacuo to give a colorless oil. The oil was dissolved in methylene chloride and purified on silica gel eluting with 10% methanol in ethyl acetate affording Compound 32 (0.400 g, 85%) as a white solid: MS (M+1, 310)

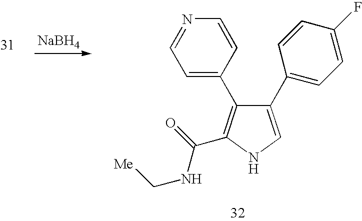

### EXAMPLE 33

### 3-(4-Fluorophenyl)-1-methyl-4-(4-pyridinyl)-1H-pyrrole-2-carboxylic acid (Compound 33)

A solution of potassium hydroxide (0.862 g, 15.4 mmol) in water (10 mL) was added to Compound 2 (0.500 g, 1.54 mmol) dissolved in methanol (10 mL). The reaction mixture was stirred at reflux for 2 hours, then concentrated in vacuo. The aqueous layer was extracted with ethyl acetate (1×10 mL). The pH was then adjusted to neutral using solid ammonium chloride, then extracted with ethyl acetate (3×25 mL) and the organic extracts were dried over sodium sulfate. The drying agent was filtered and solvent removed in vacuo to afford Compound 33 (0.391 g, 86%). ¹H NMR (CDCl₃) δ 8.35 (2H, d), 7.2 (2H, m), 7.15 (1H, s), 7.05 (2H, t), 6.95 (2H,d), 4.0 (3H, s).

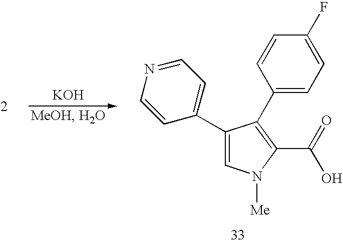

### EXAMPLE 34

### 3-(4-Fluorophenyl)-1-methyl-N-(2-oxo-2-phenylethyl)-4-(4-pyridinyl)-1H-pyrrole-2-carboxamide (Compound 34)

Compound 33 (0.400 g, 1.35 mmol), 2-aminoacetophenone hydrochloride (0.301 g, 1.75 mmol), 1-(3-dimethylaminopropyl)-3-ethylcarbodiimide hydrochloride (0.647 g, 3.37 mmol), 1-hydoxybenzotriazole hydrate (0.456 g, 3.37 mmol), triethylamine (0.491 g, 4.85 mmol) were dissolved in N,N-dimethylformamide (8 mL) and the mixture was allowed to stir at room temperature for 16 hours. The reaction mixture was then diluted with water (90 mL) and the resulting solid was filtered, washed with water and allowed to air dry on the vacuum filter to afford the desired product Compound 34. The filtrate was then extracted with ethyl acetate (3×30 mL), washed with brine (1×30 mL) and dried over sodium sulfate. The drying agent was filtered and solvent removed in vacuo to afford Compound 34 as a solid. The combined solids were purified using column chromatography to afford the desired Compound 34 (0.366 g, 66%): ¹H NMR (CDCl₃) δ 8.40 (2H, bs), 7.9 (2H, d), 7.6 (1H, t), 7.6 (2H, t), 7.3 (2H, m), 7.18 (2H, t), 7.05 (1H, d), 6.95 (2H, bs), 6.35 (1 H, bs), 4.7 (2H, d), 4.0 (3H, s).

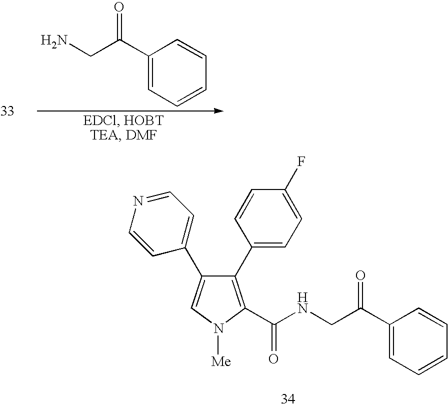

### EXAMPLE 35

### 4-[4-(4-Fluorophenyl)-1-methyl-5-(1,3,4-oxadiazol-2-yl)-1H-pyrrol-3-yl]pyridine (Compound 35)

Compound 35a (62.0 mg, 0.183 mmol) (Example 34 synthesis) was dissolved in anhydrous acetonitrile (10 mL) and carbon tetrachloride (0.8 mL). Triethylamine (0.2 mL), triphenyl phosphine (144.0 mg) were added sequentially at room temperature and the resulting mixture was stirred for 2 hours. The reaction mixture was quenched with water, extracted with ethyl acetate twice and washed with sodium chloride solution. The organic layers were separated, dried (anhydrous sodium sulfate), filtered and concentrated in vacuo to give an oily residue. The residue was purified with column chromatography to afford Compound 35 (38.1 mg, 65%) as a white solid: ¹H NMR (CDCl₃) δ 8.15˜8.4 (3H, m), 6.8˜7.20 (7H, m), 4.05 (3H, s).

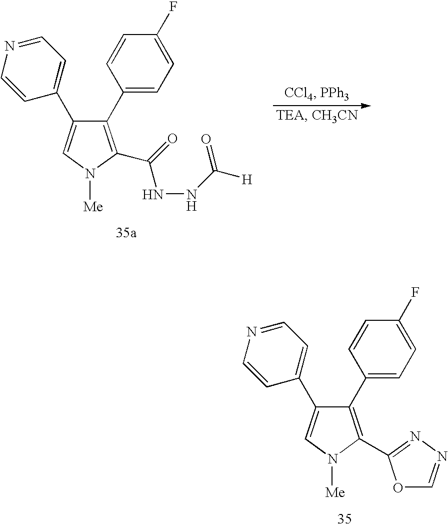

### EXAMPLE 36

### 4-[4-(4-Fluorophenyl)-1-methyl-5-(5-phenyl-2-oxazolyl)-1H-pyrrol-3-yl]pyridine (Compound 36)

Trichloroacetyl chloride (0.027 mL, 0.242 mmol) and pyridine (0.040 mL, 0.484 mmol), were added to a solution of Compound 34 (0.050 g, 0.121 mmol) in dichloromethane (1 mL). The reaction mixture was allowed to stir at 25° C. for 1 hour, diluted with dichloromethane (10 mL), then washed with aqueous saturated sodium bicarbonate (5 mL), 1N citric acid (5 mL), water (5 mL), brine (10 mL) and dried over sodium sulfate. The drying agent was filtered and solvent removed in vacuo to afford a brown solid. The solid was purified on a preparatory thin layer chromatography plate to afford the desired Compound 36 (0.0263 g, 55%): ¹H NMR (CDCl₃) δ 8.35 (2H, d), 7.3 (5H, m), 7.2 (2H, d), 7.1 (3H, m), 7.0 (2H, d), 4.15 (3H, s).

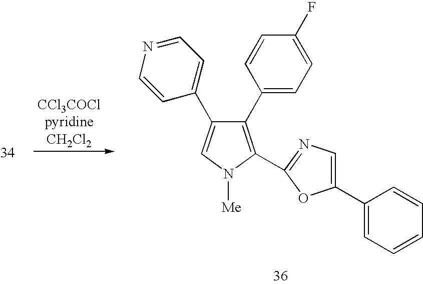

### EXAMPLE 37

### 3-(4-Fluorophenyl )-N-methoxy-N,1-dimethyl-4-(4-pyridinyl)-1H-pyrrole-2-carboxamide (Compound 37)

Isopropylmagnesium chloride (5.0 mL, 10.1 mmol) was added slowly to a cooled (−10° C.) solution of Compound 3 (0.500 g, 1.54 mmol) and N,O-dimethylhyroxylamine hydrochloride (0.496 g, 5.08 mmol) in tetrahydrofuran (10 mL). The reaction mixture was stirred for 0.75 hours, then quenched with saturated aqueous ammonium chloride (10 mL) and extracted with ethyl acetate (3×20 mL). The organic extracts were dried over sodium sulfate, filtered and concentrated in vacuo to afford the desired Compound 37 (0.507 g, 97%): ¹H NMR (CDCl₃) δ 8.40 (2H, d), 7.15 (2H, m), 7.0 (5H, m), 3.75 (3H, s), 3.45 (3H, s), 2.95 (3H, s).

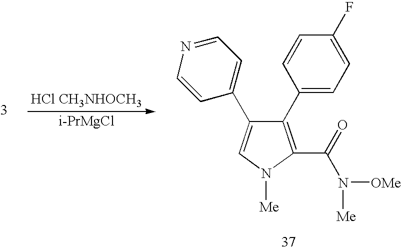

### EXAMPLE 38

### 3-(4-Fluorophenyl)-1-methyl-4-(4-pyridinyl)-1H-pyrrole-2-carboxaldehyde (Compound 38)

A solution of Compound 37 (0.500 g, 1.47 mmol) in tetrahydrofuran (15 mL) was added slowly to lithium aluminum hydride (8.0 mL, 8.0 mmol) at −78° C. and the mixture was stirred for 1.5 hours. The reaction mixture was quenched slowly with sodium sulfate decahydrate and allowed to stir overnight. The resulting solids were filtered then washed with tetrahydrofuran. The organics were concentrated in vacuo to an oily residue. The residue was purified on a preparatory thin layer chromatography plate to afford the desired Compound 38 (0.317 g, 77%): ¹H NMR (CDCl₃) δ 9.45 (1H, s), 8.40 (2H, d), 7.3 (1H, s), 7.15 (4H, m), 7.0 (2H, d), 4.1 (3H, s).

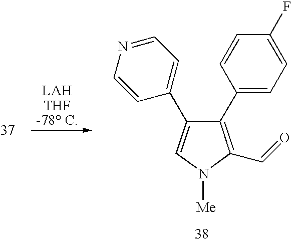

### EXAMPLE 39

### 1-[3-(4-Fluorophenyl)-1-methyl-4-(4-pyridinyl)-1H-pyrrol-2-yl]ethanone (Compound 39)

Compound 37 (98.1 mg, 0.289 mmol) was dissolved in tetrahydrofuran (3 mL) and methyl magnesium bromide (3.0 M in ether, 0.1 mL) was added at 0° C. and the mixture was stirred for 4 hours at room temperature. Additional MeMgBr solution (0.6 mL) was added in three portions over the next 48 hours. The reaction mixture was quenched with aqueous ammonium chloride and allowed to stir for 5 minutes. The resulting mixture was extracted with ethyl acetate twice and the organic layers were washed with sodium chloride solution, dried with anhydrous sodium sulfate, filtered and concentrated in vacuo to an oily residue. The residue was purified with column chromatography to afford the desired Compound 39 (57.5 mg, 71%): ¹H NMR (CDCl₃) δ 8.35 (2H, m), 6.90˜7.25 (7H, m), 4.00 (3H, s), 1.89 (3H, s).

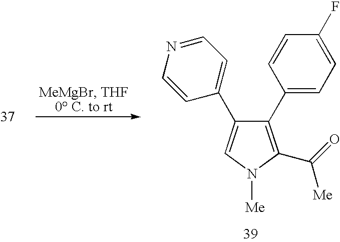

### Example 40

### 3-(4-Fluorophenyl)-□, 1-dimethyl-4-(4-pyridinyl)-1H-pyrrole-2-methanol (Compound 40)

Compound 39 (32.1 mg, 0.115 mmol) was dissolved in absolute ethanol (1.5 mL) and sodium boron hydride (15.0 mg) was added in one portion at room temperature and the resulting mixture was stirred for 2 hours, quenched with water then allowed to stir for 10 minutes. The mixture was extracted with ethyl acetate twice. The organic extracts were washed with sodium chloride solution, dried with anhydrous sodium sulfate, filtered and concentrated in vacuo to an oily residue. The residue was purified with column chromatography to afford the desired Compound 40 (31.0 mg, 96%) as a white solid: ¹H NMR (CDCl₃) δ 8.25 (2H, m), δ 6.85˜7.20 (7H, m), 4.96 (1 H, q), 3.90 (3H, s), 2.20 (1H, br), 1.57 (3H, d).

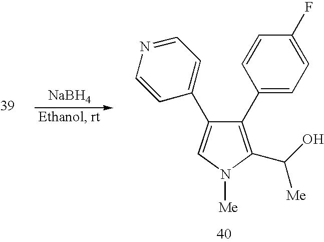

### EXAMPLE 41

### 4-[4-(4-Fluorophenyl)-1-methyl-5-(5-oxazolyl)-1H-pyrrol-3-yl]pyridine (Compound 41)

Compound 38 (0.025 g, 0.089 mmol), tosylmethyl isocyanide (0.027 g, 0.143 mmol) and potassium carbonate (0.019 g, 0.143 mmol) were stirred at reflux in methanol (2.5 mL) for 2.5 hours. The reaction mixture was cooled, diluted with ethyl acetate (10 mL), then washed with water (2×5 mL), brine (1×10 mL) and dried over sodium sulfate. The drying agent was filtered and solvent removed in vacuo to afford the desired Compound 41 (0.028 g, 98%) as a light yellow solid: ¹H NMR (CDCl₃) δ 8.35 (2H, d), 7.9 (1 H, s), 7.1 (3H, m), 7.0 (4H, m), 6.75 (1 H, s), 3.75 (3H, s).

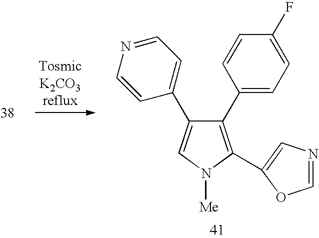

### EXAMPLE 42

### 5-Bromo-4-(4-fluorophenyl)-3-(4-pyridinyl)-1H-pyrrole-2-carboxylic acid methyl ester (Compound 42)

Compound 25 (0.592 g, 2 mmol) was mixed with anhydrous methylene chloride (15 mL) and NBS (0.463 g, 2.6 mmol) was portionwise added at 0° C. After 5 minutes, the precipitates were collected by filtration to give the product Compound 42 (0.502 g). The filtrate was diluted with water and methylene chloride. The organic layer was washed with water then brine, dried (anhydrous potassium carbonate), filtered through Celite and concentration in vacuo to give the crude product. Purification by chromatography (silica gel, 50% ethyl acetate/hexanes) yielded an additional crop of Compound 42 (0.1 g). The total yield of Compound 42 was 80%. MS (M+1, 375)

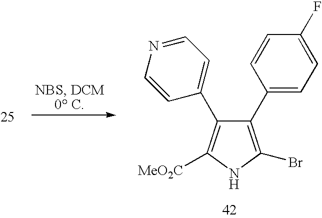

### EXAMPLE 43

### 5-Bromo-4-(4-fluorophenyl)-1-methyl-3-(4-pyridinyl )-1H-pyrrole-2-carboxylic acid methyl ester (Compound 43)

Sodium hydride (60% in mineral oil, 14.1 mg, 0.352 mmol) was added portionwise to a solution of Compound 42 (0.12 g, 0.32 mmol) in DMSO (2 mL) under N₂. The mixture was stirred for 10 minutes at room temperature then methyl iodide (0.022 mL, 0.352 mmol) was added. The reaction mixture was stirred at room temperature for 25 minutes and then quenched with water (10 mL). The yellow precipitate was collected by filtration, washed with water and dried under vacuum to give Compound 43 (0.101 g, 81%). MS (M+1, 390)

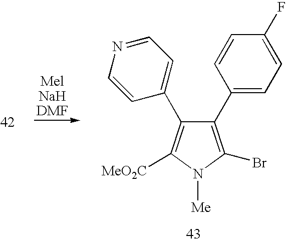

### EXAMPLE 44

### 4-(4-Fluorophenyl)-1-methyl-5-[3-(4-morpholinyl)-1-propynyl]-3-(4-pyridinyl)-1H-pyrrole-2-carboxylic acid methyl ester (Compound 44)

A mixture of Compound 43 (0.243 g, 0.625 mmol), N-propargyl morpholine (0.195 g, 1.562 mmol), Pd(PPh₃)₂Cl₂ (10 mol%, 43.9 mg), PPh₃ (5 mol%, 8.2 mg) and TEA (1.875 mmol, 0.26 mL) in anhydrous THF (3 mL) was degassed and stirred at room temperature for 10 minutes. Cul (2.4 mol%, 2.9 mg) was added and the resulting mixture was heated to 60° C. under N₂ and monitored by TLC. After 16 hr, the reaction mixture was cooled to room temperature and diluted with water (10 mL) and ethyl acetate (20 mL). The aqueous layer was extracted with ethyl acetate (2×15 mL) and the combined organic layers were washed with water, brine and dried over anhydrous potassium carbonate. Filtration through Celite and concentration in vacuo gave the crude product. Purification by chromatography (silica gel, methanol/ethyl acetate) yielded Compound 44 (0.17 g, 63%). MS (M+1, 434)

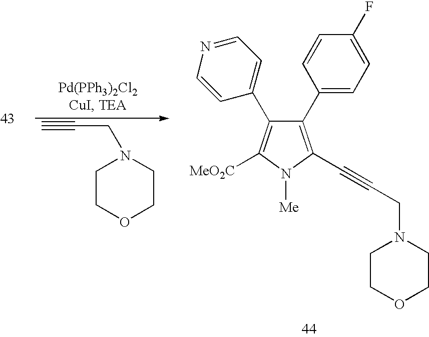

### EXAMPLE 45

### 3-(4-Fluorophenyl )-1-(2-hydroxyethyl)-4-(4-pyridinyl )-1H-pyrrole-2-carboxylic acid (Compound 45)

A mixture of Compound 2 (3.0 g, 0.0101 mol), 2-bromoethylacetate (1.34 mL, 0.0122 mol) and cesium carbonate (3.96 g, 0.0122 mol) in DMF (50 mL) was heated to 55° C. for 7 h. The reaction mixture was cooled to rt, quenched with H₂O (150 mL) and extracted with EtOAc (3×150 mL). The combined organic layers were washed with H₂O (1×50 mL), brine (1×50 mL), dried over sodium sulfate, filtered and concentrated under reduced pressure. The resulting crude product 45a was used for the next step without further purification.

A mixture of Compound 45a and NaOH (2 N, 50 mL, 0.101 mol) in methanol (250 mL) was heated to 65° C. overnight, concentrated and the resulting residue was diluted with H₂O (200 mL) and extracted with EtOAc (3×100 mL). The aqueous layer was neutralized with saturated NH₄Cl solution and extracted with EtOAc (3×400 mL). The combined organic layers were dried over MgSO₄ and concentrated. The resulting residue was triturated with ether and the precipitate was collected by filtration and dried under vacuum to give Compound 45 (2.40 g, 73% yield for 2 steps): ¹H NMR (300 MHz, DMSO) δ 8.33-8.28 (2H, d), 7.62 (1H, s), 7.23-7.10 (4H, m), 6.94-6.90 (2H, d), 4.47-4.41 (2H, t), 3.77-3.73 (2H, t).

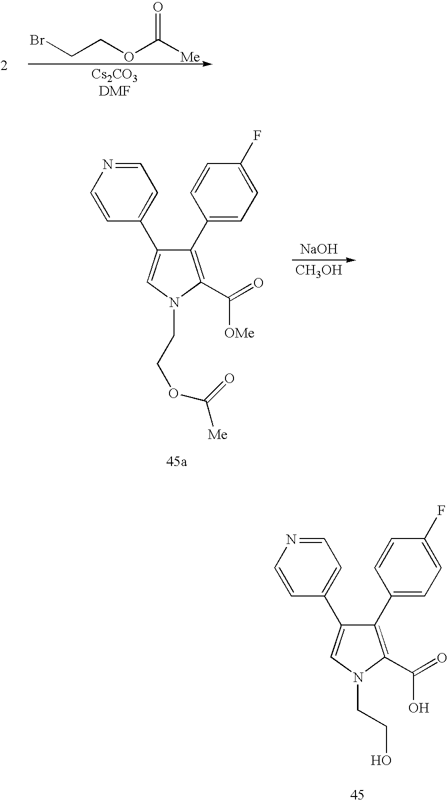

### EXAMPLE 46

### 8-(4-Fluorophenyl)-3,4-dihydro-7-(4-pyridinyl)-1H-pyrrolo[2,1-c][1,4]oxazin-1-one (Compound 46)

Compound 45 (0.050 g, 0.153 mmol) and 1-[3-(dimethylamino)propyl]-3-ethylcarbodiimide hydrochloride (EDC) (0.035 g, 0.184 mmol) were mixed with DMF (5 mL) and i-Pr₂EtN (0.060 mL, 0.337 mmol) was added at rt. The reaction mixture was stirred overnight and quenched with H₂O (20 mL) then extracted with EtOAc (3×50 mL). The combined organic layers were dried over MgSO₄ and filtered. The solvent was removed under reducing pressure and the resulting residue was triturated with ether. The precipitate was filtered and dried under vacuum to give Compound 46 (23 mg, 49%): ¹H NMR (300 MHz, DMSO) δ 8.50-8.47 (d, 2H), 7.73 (1H, s), 7.27-7.14 (4H, m), 7.00-6.96 (2H, d), 4.67-4.63 (2H, t), 4.41-4.35 (2H, t).

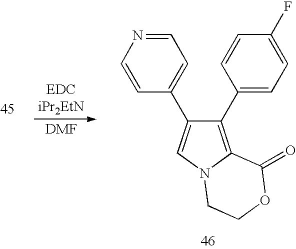

### EXAMPLE 47

### 6-(4-Fluorophenyl)-2,3-dihydro-1-methyl-7-(4-pyridinyl)-1H-pyrrolizine-5-carboxylic acid methyl ester (Compound 47)

A solution of Compound 47a (0.0422 g, 0.0983 mmol) (example 4 synthesis) and AIBN (0.005 g, 0.0295 mmol) in toluene (8.0 mL) was heated to reflux and a solution of Bu₃SnH (1 N, 0.0661 mL, 0.246 mmol) in toluene (4 mL) was added over a period of 10 min. The mixture was refluxed overnight then cooled to rt and concentrated under vacuum. The resultant liquid was treated with EtOAc (6 mL), KF (0.256 g) and H₂O (0.233 mL). This solution was stirred for 0.5 h at rt. Potassium carbonate was then added to the mixture and the solids were filtered off. The resultant solution was concentrated in vacuo and the residue was chromatographed on silica gel (ethyl acetate/hexane =6:4) to afford Compound 47 (0.017 g, 52%). MS (M+1, 351) 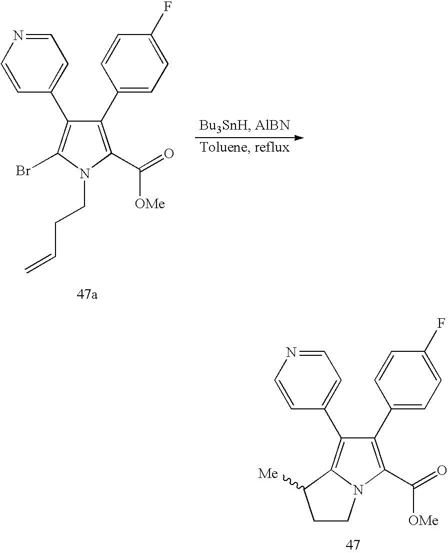
Biological Assays and Activity

A. p38 Inhibition In-Vitro Enzyme Assays

Assay 1

Purified recombinant 6×His-p38 and GST-ATF2 (both expressed in Baculovirus) were served as enzyme and substrate in our p38 inhibition assay. The enzyme reaction was performed in the anti-GST antibody pre-coated 96-well ELISA plate.

A 96-well ELISA plate was coated with 100 μl/well of anti-GST antibody (from Amersham) at a concentration of 2 μ/ml and incubated overnight at 4° C. The ELISA plate was blocked with 100 μl/well of 1% BSA at 25° C. for at least 2 hours, then the plate was washed with 300 μl PBST. The enzyme reaction was started by adding 10 μl of p38 recombinant protein (0.05 nM in final concentration), 10 μl of compound in desired concentration, 10 μl of substrates (670 μM ATP, 200 nM GST-ATF2 in final concentration) and 70 μl of reaction buffer (50 mM HEPES, pH 7.4, 100 mM NaCl, 10 mM MgCl₂) and the mixture was incubated at 37° C. for 30 min. Washing the reaction plate with 3×300 μl/well of PBST stopped the enzyme reaction. Detection of the phosphorylated ATF2 substrate was done by the addition of 100 μl of anti-Pi-ATF2 antibody (from Cell Signaling) at a 1:200 dilution. The mixture was incubated at 25° C. for 30 min. A solution of 100 μl of HRP-conjugated goat anti-rabbit antibody (from Pierce) at a 1:250 dilution was added and the mixture and incubated at 25° C. for 30 min. The plate was washed with 6×300 μI PBST and 100 μl of OPD substrate (from Sigma) was then added. The mixture was incubated at 25° C. for 10 min. before adding 100 μl of 3 N H₂SO₄. The plate was read at 490 nm.

The % inhibition of each test compound was calculated by the following formula: % inhibition=[1−(sample−BKG)/(CTRL−BKG)]×100. The IC50 of each test compound was determined by a triplicate-8 point dose titration curve by Prism GraphPad program.

Assay 2

A solution (38 μL) of purified recombinant p38 (6×His-p38 expressed in E.coli), myelin basic protein substrate (determined empirically), and a buffer of pH 7.5 (Hepes:25 mM, MgCl2:10 mM, MnCl2:10 mM) were added to 92 wells of a 96-well round bottom polypropylene plate. The amount of enzyme was determined empirically based on the linear range of the assay and the acceptable signal to noise ratio. The remaining wells were used for control (“CTRL”) and background (“BKG”). The CTRL was prepared with the enzyme, substrate buffer and 2% DMSO, and the BKG was prepared with substrate buffer and 2% DMSO.

A solution (12 μL) of the test compound in DMSO was added to the testing wells. Compounds were diluted to 125 μM in 10% DMSO/H₂O and assayed at 25 μM where the final DMSO concentration was 2%. The ATP/³³P-ATP solution (10 μL containing 50 μM unlabeled ATP and 1 μCi ³³P-ATP) was added to all wells and the completed plates were mixed and incubated at 30° C. for 30 min. Ice-cold 50% TCA/10 mM sodium phosphate (60 μL) was added to each well and the plates were kept on ice for 15 min. The contents of each well were transferred to the wells of a 96-well filterplate (Millipore, MultiScreen-DP) and the filterplate was placed on a vacuum manifold fitted with a waste collection tray. The wells were washed five times with 10% TCA/10 mM sodium phosphate (200 μL) under vacuum. MicroScint-20 scintillant was added, and the plates were sealed using Topseal-S sheets and counted in a Packard TopCount scintillation counter using a ³³p liquid program with color quench correction, where the output is in color quench-corrected cpm. The % inhibition of each test compound was calculated by the following formula: % inhibition=[1−(sample−BKG)/(CTRL−BKG)]×100.

Although compounds were initially tested at 1 μM, when warranted, the compounds were also tested at 4-fold increments above and below that concentration. In addition, the IC⁵⁰ was calculated for some compounds using the Deltagraph 4-parameter curve-fitting program.

B. In-Vitro Whole Cell Assay for TNF-□ Inhibition

Freshly obtained venous blood was anticoagulated with heparin, diluted with an equal volume of phosphate buffered saline (“PBS”) and placed in a sterile tube or other container. Aliquots (30 mL) of this mixture were transferred to centrifuge tubes, which were underlaid with Ficoll-Hypaque (15 mL). The prepared tubes were centrifuged at 400×g, without braking, for 30 min at room temperature. Approximately ½ to ⅔ of the platelet layer above the mononuclear cell band was removed with a pipette. The majority of the mononuclear cell layer was carefully removed using a pipette and these PBMC's were diluted with PBS and spun at 600×g for 15 min. The resulting PBMC's were washed with another portion of PBS and spun at 400×g for 10 min at room temperature. The recovered pellets were diluted in low endotoxin RPMI/1% FCS culture medium, and gave a cell concentration of 0.5-2.0×10⁶ PMBC/ mL. A small volume of the suspension was removed for counting on a hemocytometer and the remaining preparation was centrifuged at 200×g for 15 min at room temperature. The recovered pelleted PMBC's were resuspended in RPMI/1% FCS to a concentration of 1.67×10⁶/mL.

To run the assay, the PBMC suspension (180 μL) was transferred to duplicate wells of a 96-well flat-bottom microtiter plate and incubated for 1 h at 37° C. A solution of test compound (10 μL: prepared at 20× the desired final concentration) was added to each well and the plate was incubated for 1 h at 37° C. A solution (10 μL) of LPS in RPMI/1% FCS (200 ng/mL) was added and the wells were incubated overnight at 37° C. The supernate (100 μL) was removed from each well and diluted with RPMI/1% FCS (400 μL). The samples were analyzed for TNF-α using a commercial ELISA kit (Genzyme).

C. In Vivo Rodent Assay for Inhibition of TNF-□ Production

The ability of the instant compounds to inhibit LPS-induced TNF-α production was demonstrated in the following in vivo rodent assays. Mice (BALB/cJ females, Jackson Laboratories) or rats (Lewis males, Charles River) were fasted for 30 min. The animals were dosed orally on a mg/kg basis at a range of times prior to being injected intraperitoneally with LPS at 1 mg/kg and returned to their cages for 1 h. Animals were anesthetized by CO₂, exsanguinated by cardiac puncture and whole blood collected (0.1-0.7 mL). The blood was allowed to clot and serum was transferred to a centrifuge tube. This sample was centrifuged, and serum was collected, aliquoted and frozen at −80° C. Samples were tested by commercial ELISA's for TNF-α (Endogen for mouse TNF-α and Biosource for rat TNF-α). The compounds were tested for their ability to inhibit TNF-α production in mice and the data are listed as % inhibition at 10 mg/kg.

1. A compound of Formula I 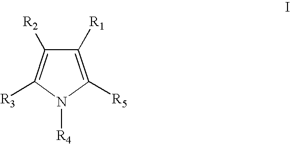
or a pharmaceutically acceptable salt thereof, wherein
R₁ and R₂ are independently selected from optionally substituted aryl and optionally substituted heteroaryl;
R₃ is selected from hydrogen, optionally substituted alkyl, —N═CR′″, —C(O)R′, —C(O)NR′R″, —NR′R″, optionally substituted aryl, optionally substituted heteroaryl, and optionally substituted heterocycle, wherein R′ and R″ are independently selected from hydrogen, optionally substituted alkyl, optionally substituted aryl, and optionally substituted heterocycle;
R₄ is selected from hydrogen, optionally substituted alkyl, optionally substituted aryl, optionally substituted heteroaryl, optionally substituted heterocycle, and —SiR′″R″″R′″″ wherein R′″, R″″, and R′″″ are each an independent straight chain or branched C₁₋₅alkyl, or R₃, R₄ and the —C—N— to which R₃ and R₄ are connected together form an optionally substituted 5- or 6-membered ring;
R₅ is selected from optionally substituted alkyl, —C(O)OR′, —C(O)NR′R″, C(O)NHNHC(O)R₆, —SO₂NR′R″, —C(O)R′, —NR′R″, nitrile, nitro, halo, and optionally substituted heterocycle, or R₄, R₅ and the —C—N— to which R₄ and R₅ are connected together form an optionally substituted 5- or 6-membered ring;
R₆ is selected from H, alkyl, optionally substituted aryl; and
with the provisos that
(1) R₁ and R₂ are not both optionally substituted phenyl;
(2) if either R₁ or R₂ is optionally substituted phenyl or 3-thienyl, and the other is unsubstituted 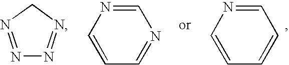
then R₃is not hydrogen, unsubstituted alkyl, —(CH₂)₃OH, —(CH₂)₃PH, —(CH₂)₃OMs, or —(CH₂)₂N(CH₂)₂O(CH₂)₂, and R₅ is not unsubstituted alkyl, —(CH₂)₃OH, —(CH₂)₃PH, —(CH₂)₃OMs, or —(CH₂)₂N(CH₂)₂O(CH₂)₂; and
(3) R₄ doesn't form a fused ring with both R₃ and R₅.

2. The compound of claim 1 wherein R₁ is substituted with a group selected from hydrogen, amino, alkyl substituted amino, aryl substituted amino, hydroxy, methoxy, phenyl ether, S-alkyl, halogen, trifluoromethyl, and nitro.

3. The compound of claim 1 wherein R₂ is substituted with a group selected from hydrogen, amino, alkyl substituted amino, aryl substituted amino, hydroxy, methoxy, phenyl ether, S-alkyl, halogen, trifluoromethyl, and nitro.

4. The compound of claim 3 wherein R₂ is heteroaryl having 1-3 N.

5. The compound of claim 1 wherein R₃ is selected from hydrogen, alkyl, aryl, heteroaryl, heterocycle, and —NR′R″, wherein R′ and R″ are independently selected from hydrogen, alkyl, aryl, and heterocycle.

6. The compound of claim 1 wherein R₄ is hydrogen or alkyl.

7. The compound of claim 6 wherein R₄ is hydrogen or methyl.

8. The compound of claim 1 wherein R₅ is selected from alkyl, —C(O)OR′, —C(O)NR′R″, nitrile, and heterocycle.

9. The compound of claim 8 wherein R₅ is selected from is selected from —(CH₂)ₙOR′, —(CH₂)ₙNR′R′″, —(CH₂)ₙCOOR′, —(CH₂)ₙCONR′R″, —NHCOR′, and ester isosteres.

10. The compound of claim 9 wherein R₅ is selected from oxadiazole, 1,2,4-triazole, 1,2,4-triazol-3-ol, isoxazol-3-ol, imidazolidine-2,4-dione, 4H-[1,2,4]thiadiazol-5-one, oxazole, and [1,3,4]oxadiazole.

11. The compound of claim 10 wherein R₅ is 4H-[1,2,4]oxadiazole-5-thione or 4H-[1,2,4]oxadiazol-5-one.

12. The compound of claim 1 having the structure

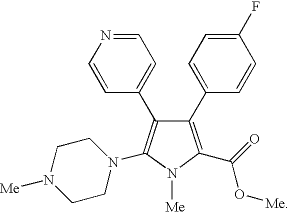

13. The compound of claim 1 having the structure

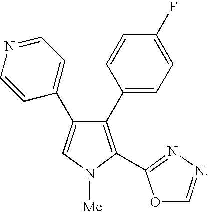

14. The compound of claim 1 having the structure

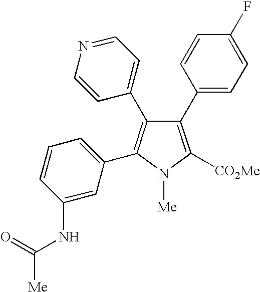

15. The compound of claim 1 having the structure

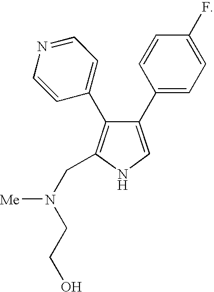

16. The compound of claim 1 having the structure

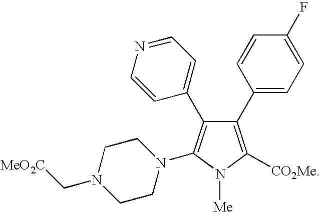

17. The compound of claim 1 having the structure

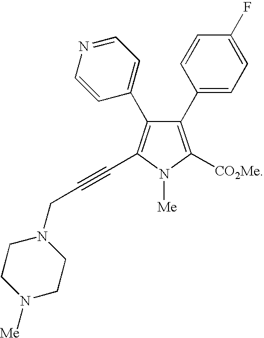

18. The compound of claim 1 having the structure

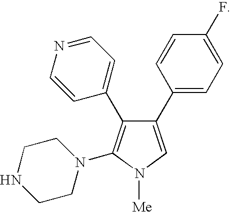

19. The compound of claim 1 having the structure

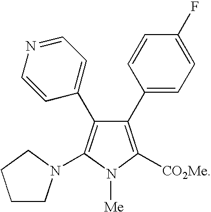

20. The compound of claim 1 having the structure

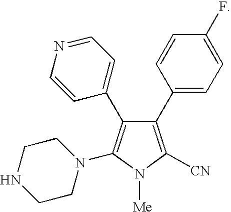

21. The compound of claim 1 having the structure

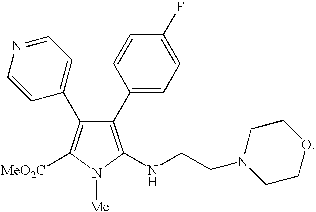

22. The compound of claim 1 having the structure

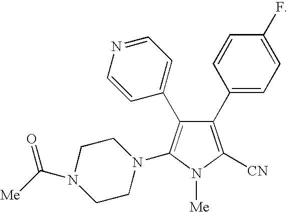

23. The compound of claim 1 having the structure

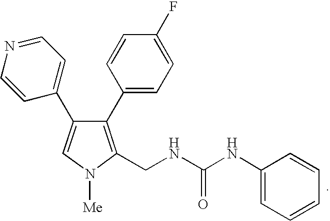

24. A pharmaceutical composition comprising the compound of claim 1 and a pharmaceutically acceptable carrier.

25. A method of treating a subject having a disorder ameliorated by reducing TNF-α production and/or p38 activity in appropriate cells, which comprises administering to the subject a therapeutically effective dose of the pharmaceutical composition of claim 24.

26. The method of claim 25, wherein the disorder is an inflammatory disorder.

27. The method of claim 25, wherein the disorder is selected from the group consisting of rheumatoid arthritis, osteoporosis, osteoarthritis, allergic inflammation, periodontal disorder, inflammatory bowel disorder, septic shock, insulin-dependent diabetes mellitus, non-insulin-dependent diabetes, cachexia, pulmonary fibrosis, myasthenia gravis, Crohn's disease, hepatitis, primary biliary cirrhosis, acute pancreatitis, allograph rejection, glioblastoma, alopecia areta, psoriasis, ischemia, congestive heart failure, restenosis, atherosclerosis, systemic lupus erythematosus, nephritis, Guillain-Barre Syndrome, viral myocarditis, HIV replication, T-cell depletion in HIV infection, cognitive deficits induced by neuronal inflammation, multiple sclerosis, stroke, neuropathic pain, HIV dementia and Alzheimer's disease.

28. The method of claim 27, wherein the disorder is rheumatoid arthritis.

29. A method of preventing an inflammatory response in a subject, comprising administering to the subject a prophylactically effective amount of the pharmaceutical composition of claim 24 either preceding or subsequent to an event anticipated to cause the inflammatory response in the subject.

30. The method of claim 29, wherein the event is selected from the group consisting of an insect sting and an animal bite.
